(19) United States
(12) Patent Application Publication
(10) Pub. No.: US 2016/0017268 A1
(43) Pub. Date: Jan. 21, 2016

(54) DEVICE AND METHODS COMPRISING MICROELECTRODE ARRAYS FOR ELECTROCONDUCTIVE CELLS

(71) Applicants: UNIVERSITY OF WASHINGTON THROUGH ITS CENTER FOR COMMERCIALIZATION, Seattle, WA (US); ARIZONA BOARD OF REGENTS FOR AND ON BEHALF OF ARIZONA STATE UNIVERSITY, Scottsdale, AZ (US)

(72) Inventors: Deok-Ho KIM, Seattle, WA (US); Michael LAFLAMME, Seattle, WA (US); Junseok CHAE, Scottsdale, AZ (US); Kshitiz GUPTA, Seattle, WA (US)

(21) Appl. No.: 14/774,059

(22) PCT Filed: Mar. 14, 2014
(86) PCT No.: PCT/US14/28530

§ 371 Date: Sep. 9, 2015

Related U.S. Application Data
(60) Provisional application No. 61/788,365, filed on Mar. 15, 2013.

(51) Int. Cl. C12M 1/42; C12M 1/34

(52) U.S. Cl. CPC C12M 35/02; C12M 41/46

(57) ABSTRACT

The present invention relates to the devices and method comprising microelectrode arrays for the differentiation, maturation and functional analysis of electroconductive cells, including muscle cells (including, but not limited to, cardiomyocytes, skeletal muscle myocytes and smooth muscle myocytes) and neuronal cells. The microelectrode present on the arrays can be used to stimulate and record from cells cultured on the substrate. In some embodiments, the substrate has a substantially smooth surface, and in other embodiments the substrate is nanotextured, including an array of substantially parallel grooves and ridges of nanometer-micrometer widths.

### CROSS-REFERENCE TO RELATED APPLICATIONS

This application claims benefit under 35 U.S.C. §119(e) of U.S. Provisional Application No. 61/788,365 filed Mar. 15, 2013, the contents of which are incorporated herein by reference in its entirety.

### TECHNICAL FIELD

The present invention generally relates to the field of cell growth and tissue engineering, in particular, a device comprising microelectrodes for the differentiation, maturation and functional analysis of electroconductive cells, including muscle cells and neuronal cells.

### BACKGROUND

The culture of electroconductive cells, e.g. neurons and muscle cells, in a manner that permits measurement of their electroconductive behavior is important for understanding the biology of these cells, determining if candidate therapeutics perturb this behavior (possibly leading to dangerous side effects in patients), and identifying agents that can therapeutically modify electroconductive behaviors, e.g. for the treatment of arrhythmia. However, the existing technologies for culturing these cells suffer from suboptimal spatial accuracy, low-throughput, and alter the natural physiology and/or behavior of electroconductive cells due the characteristics of the culture environment.

### SUMMARY

The compositions and methods described herein relate to devices for the culture of electroconductive cells which can flexibly provide electrical stimulation or data collection capabilities as well. As demonstrated herein, these devices support the growth of electroconductive cells which exhibit behaviors which are more physiologically relevant than prior methods allowed (e.g. increased force generation and/or increased field potentials). Additionally, the devices provided herein allow high-throughput stimulation and data collection with a resolution not previously possible.

The present invention generally relates to devices and methods comprising microelectrode arrays for the differentiation, maturation and functional analysis of electroconductive cells, including muscle cells (including, but not limited to, cardiomyocytes, skeletal muscle myocytes and smooth muscle myocytes) and neuronal cells. The microelectrode arrays present on or adjacent to the cell culturing surface of the device can be used to stimulate and record from cells cultured on the surface. In some embodiments, the cell culturing surface has a substantially smooth surface, and in other embodiments the cell culturing surface can be nanotextured, including an array of substantially parallel grooves and ridges of nanometer and/or micrometer widths.

In some embodiments, the electrodes present on the microelectrode array (MEA) device (referred to herein as a “MEA device”) are in contact with the cultured cells on the cell culture surface, and can be used to acquire biopotentials from the cultured cells. Leads from the microelectrodes can connect the microelectrodes to the edge of the device to transfer the signals produced by the cultured cells (e.g., biopotentials) from the cells to the signal recording and/or processing electronics. In alternative embodiments, the leads which connect to the microelectrodes which are in contact with the cultured cells can transfer a signal (e.g., an electrical input) from a signal generator to the cultured cells. In some embodiments, the signal generator can provide an input signal which has a predefined shape and amplitude, (e.g, a sine wave, a square wave or saw-tooth wave).

In some embodiments, the leads which connect the microelectrodes to the edge of the device can be selectively covered with a non-conducting, insulating or dielectric material (e.g., to prevent the cultured cells which come in contact with the leads from injecting unwanted biopotentials to the signal path or receiving unintended input signals). In some embodiments, the non-conductive material can be parylene or other non-conductive, insulating or dielectric material, which are known by persons of ordinary skill in the art.

In some embodiments, the device comprises a plurality of microelectrodes on or adjacent to the cell culturing surface, e.g., at least about 2, or at least about 3, or at least about 4, or at least about 5, or at least about 6, or at least about 7, or at least about 8, or at least about 9, or at least about 10, or more than 10 microelectrodes on or adjacent to the cell culturing surface. In some embodiments, the microelectrodes can be of any geometry or shape commonly known to persons of ordinary skill in the art. In some embodiments, the microelectrodes can be arranged in a predefined pattern (e.g., circular, oval, elliptical, rectangular, square pattern) or an array. In some embodiments, the microelectrodes can be arranged in a random or pseudo-random configuration. In some embodiments, some of the microelectrodes can be arranged in a predefined pattern or array and some of the microelectrodes can be randomly located within the predefined pattern or array, or outside of the predefined pattern or array.

Any electrical interface to connect to the leads to the electronic systems can be used. In some embodiments, the device can be removably mounted in a socket that facilitates electrical connection between one or more microelectrodes and electronic components that can receive signals from the cultured cells or generate signals to be applied to the cultured cells. In some embodiments as demonstrated herein, an electrical signal received by a microelectrode can be transferred through an associated lead to a corresponding socket pin. The socket pin can be soldered or otherwise electrically connected to a circuit board trace or a wire that is connected to the electronic components or systems. The electronic components or systems can include a series of signal conditioning components (e.g., amplifiers and/or filters) and/or data acquisition (DAQ) components so that the signal can be digitally sampled and recorded.

In some embodiments, the data acquisition components can be connected to a computer comprising a software package for analysis of the data, e.g., LabVIEW (from National Instruments, Austin, Tex.), for visualization of the electronic signal. In some embodiments, the captured signal contains noise (e.g., low frequency or high frequency noise) which can be removed by known techniques (e.g., filtering, shielding or providing a well-grounded source) and/or software known to persons of ordinary skill in the art.

In some embodiments, the microelectrode array includes a substrate with metallization in a spectral pattern. In some embodiments, the microelectrode array can be formed by sputter coating a Cr/Au material directly on the substrate using a shadowing mask for the spectral pattern of the microelectrodes. In some embodiments, the microelectrode array can be formed in the center of a substrate with leads connecting to one or more external circuits connected through pads on the outside or edges of the substrate. The external circuits can include signal conditioning and/or filtering components as well signal multiplexing components, signal processing components, signal recording components, and/or signal generating components.

In some embodiments, the microelectrode can be used to modulate at least one biopotential of the cells cultured on the substrate, for example, but not limited to, a biopotential which is a functional parameter, including, but not limited to action potential duration (ADP), wave propagation, action potential frequency, beat frequency, action potential transmission, Vmax of the action potential, contraction force, peal to peak amplitude, end diastole to peak diastole rate and the like.

In some embodiments, the electroconductive cells include muscle cells (including, but not limited to, cardiomyocytes, skeletal muscle myocytes and smooth muscle myocytes) and neuronal cells. In some embodiments, the electroconductive cells are human cells. In some embodiments, the electroconductive cells are derived from stem cells, e.g., ES cells and/or induced pluripotent stem cells (iPSC).

In some embodiments, the MEA device comprising the electroconductive arrays can be made of a polymer known to one of ordinary skill in the art. In some embodiments, the MEA device can include biocompatible and/or biodegradable substrate or layer. In some embodiments, the MEA device can be nanofabricated from scalable biocompatible polymers, such as but not limited to polyethene glycol (PEG), polyethene glycol-gelatin methacrylate (PEG-GelMA) and chemical variants thereof and hydrogel arrays. Others include, for example, but not limited to PUA, PLGA, or PMMA.

In some embodiments, the MEA device can comprises at least one of polyglycolic acid (PGA), polylactic acid (PLA), poly(lactic-co-glycolic) acid (PLGA), polyanhydride, polycapralactone (PCL), polydioxanone and polyorthoester. One of the most common polymers used as a biomaterial is the polyester copolymer poly(lactic acid-glycolic acid) (PLGA). PLGA is highly biocompatible, degrades into biocompatible monomers (e.g., if implanted) and has a wide range of mechanical properties making this copolymer and its homopolymers, PLA and PGA, useful as a cell culturing layer for cell deposition. The layer can be porous or non-porous.

In some embodiments, other materials can be selected to be used as the cell culturing layer material, which can be selected from the group consisting of hydroxyapatite (HAP), tricalcium phosphate (TCP), tetracalcium phosphate (TTCP), dicalcium phosphate anhydrous (DCPA), dicalcium phosphate dihydrate (DCPD), octacalcium phosphate (OCP), calcium pyrophosphate (CPP), collagen, gelatin, hyaluronic acid, chitin, and poly(ethylene glycol). In alternative embodiments, the cell culturing layer can also comprise additional material, for example, but are not limited to calcium alginate, agarose, types I, II, IV or other collagen isoform, fibrin, hyaluronate derivatives or other materials (Perka C. et al. (2000) J. Biomed. Mater. Res. 49:305-311; Sechriest V F. et al. (2000) J. Biomed. Mater. Res. 49:534-541; Chu C R et. al.. (1995) J. Biomed. Mater. Res. 29:1147-1154; Hendrickson D A et al. (1994) Orthop. Res. 12:485-497).

Further, in some embodiments, the MEA device can be optically transparent, so as to facilitate observation of the cells cultured on the MEA cell culturing surface. Optical transparency can be achieved for many MEA device materials by making the structure sufficiently thin as to permit light to transmit.

In some embodiments, the cell culturing surface can comprise a substantially smooth surface. In some embodiments, the cell culturing surface can include one or more surface features that are intended to cause tissue cultured on the cell culturing surface to develop or become organized in a predefined way. In accordance with some embodiments, the surface features and the microelectrodes can be arranged in a predefined configuration to facilitate the growth of tissue on the surface in relation to the microelectrodes such that the tissue can be stimulated by input signals and studied by monitoring and recording bio potentials produced by the tissue. The configuration of the surface features which can include nanometer and micrometer sized groves and ridges and the microelectrodes can be specific to the development of the cells and tissue that are cultured on the surface.

It has been previously demonstrated that nanotextured grooves and ridges in a substrate promoted anisotropic arrangement of cardiomyocytes isolated from adult tissue (Kim et al., entitled “Nanoscale cues regulate the structure and function of macroscopic cardiac tissue constructs”, PNAS (2010) 107 (2); 565-570, and in U.S. Patent Application 61/620,301, filed on Apr. 4, 2012, and International Patent Publication WO 2013/151755; which are incorporated herein in their entirety by reference). Accordingly, in alternative embodiments, the cell culturing surface of the MEA device can comprise a nanotextured platform comprising a substantially parallel array of grooves and ridges which have a depth of about between about 10 nm-10 μm, or between about 10 nm-1000 nm, or between about 50 nm-500 nm. In some embodiments, the width of the grooves is between the range of about 50 nm-10 μm, or in the range of about 200-1000 mn, or in the range of about 5-1000 nm, where ridges, between the grooves, have a width in the range of about 50 nm-10 μm, or in the range of about 200-1000 mn, or in the range of about 5-1000 nm. In some embodiments, the width of the groove and/or ridge is between the range of 200-800 nm, and the depth of the groove (or height of the ridge) is between about 20-100 nm Methods of fabricating a cell culturing layer with nanotextured surfaces for use as a MEA device are disclosed, e.g., in the PCT Application No. PCT/US2013/032237 entitled “Systems and Methods for Engineering muscle tissue” by Kim et al., filed on Mar. 15, 2013 and published as WO 2013/151755; each of which is incorporated by reference herein in its entirety.

In some embodiments, the electroconductive cells can form a monolayer on the MEA device as disclosed herein with anisotropic and polarized cell arrangement in the direction of the nanotextures. This can be evidenced by, for example, a high spindle shape factor or major axis of cells aligned in parallel to nanotextured arrays, as detected for example, using fluorescent or other microscopy.

In some embodiments, the substantially parallel grooves and ridges present on the microelectrode (MEA) array can be fabricated over a large surface area with high fidelity and, in some embodiments, can cover a surface area greater than (>1 cm2). In some embodiments, the array of parallel grooves and ridges has a precision of texture of at least 90% fidelity, as evidenced by atomic force microscopy, and electron microscopy.

In some embodiments, the MEA device can comprise additional coatings. For example, but without wishing to be bound by theory, the cell culturing surface of an MEA device can be modified with one or more bioactive agents, deposited or adsorbed on the polymer surface, to modify cell attachment, differentiation and maturation, adhesion-dependent cell signaling and/or electroconductivity of the device. Accordingly, in some embodiments, the MEA device can include within its polymer matrix, or on its surface, a bioactive agent that enhances maturation of cardiomyocytes, enhances survival of the cultured cells in response to toxic stimuli, enhances cell adherence to the polymer layer, and/or enhances action potential wave propagation across said the layer of cultured cells on the MEA device. In some embodiments, the MEA device may comprise a substrate base layer, and optionally, a surface layer.

In some embodiments, the MEA device for use in the methods and compositions as disclosed herein can additionally provide controlled release of bioactive agents to the cultured cells, for example, growth factors and other agents to sustain or control subsequent cell growth and proliferation of the cells coated on the MEA device of the technology described herein. In such a way, the cultured cells are supplied with a constant source of growth factors and other agents for the duration of the lifetime of the cell coated scaffold. In some embodiments, the growth factors and other agents are cardiotrophic factors commonly known in the art.

Cardiotrophic factors can include, but are not limited to creatine, carnitine, taurine, cardiotropic factors as disclosed in U.S. Patent Application Serial No. 2003/0022367 which is incorporated herein by reference in its entirety, TGF-beta ligands, such as activin A, activin B, insulin-like growth factors, bone morphogenic proteins, fibroblast growth factors, platelet-derived growth factor natriuretic factors, insulin, leukemia inhibitory factor (LIF), epidermal growth factor (EGF), TGFalpha, atrial natriuretic factor, cripto, and cardiac transcription regulation factors, such as GATA-4, Nkx2.5, and Mef2-C, and products of the BMP or cripto pathway. Other cardiac enhancing peptides include cellular differentiation agents, such as cytokines and growth factors, as disclosed herein. Examples of various cell differentiation agents are disclosed in U.S. Patent Application Serial No. 2003/0022367, or Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001; each of which is incorporated by reference herein in its entirety.

In some embodiments, the MEA device can include, on its surface or embedded in the cell culturing layer matrix, poly-L-lysine, poly-D-lysine, poly-orinithine, vitronectin or erythronectin. In some embodiments, the cell culturing layer can comprise an engineered polypeptide includes including CS1, RGD, domains in extracellular matrix proteins that bind to integrin receptors, domains in extracellular matrix proteins that bind to integrin receptors, and others well known to persons of ordinary skill in the art.

In some embodiments, the MEA device comprises, either coated on its surface or within its polymer matrix, one or more agents selected from the group consisting of sphingosine phosphate or an analog thereof, fluric acid, zFADvmk, cardiotropin, or a growth factor selected from the group consisting of FGF, HGF, IGF1, SDF1a, EGF, angiopoietin, BMP, erthyropoietin (EPO), GDNG, c-GSF, GDF9, HDNF, GDF, thrombopoietin, TGFα, TGFβ, TNFα, PIGF, PDGF, interleukins IL1-IL17 and VEGF. In some embodiments, one or more agents can be selected from the group consisting of an antibody, antigen, glycoprotein, lipoprotein, DNA, RNA, polysaccharide, lipid, growth hormone, organic compound, and inorganic compound.

Additionally, in some embodiments, the surface of the MEA device can be modified to include, at least one of the agents selected from following group: (a) extracellular matrix proteins to direct cell adhesion and function (e.g., collagen, fibronectin, laminin, etc.); (b) growth factors to direct cell function specific to cell type (e.g., nerve growth factor, bone morphogenic proteins, vascular endothelial growth factor, etc.); (c) lipids, fatty acids and steroids (e.g., glycerides, non-glycerides, saturated and unsaturated fatty acids, cholesterol, corticosteroids, sex steroids, etc.); (d) sugars and other biologically active carbohydrates (e.g., monosaccharides, oligosaccharides, sucrose, glucose, glycogen, etc.); (e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins [e.g., selectins, immunoglobulins, hormones such as human chorionic gonadotropin, Alpha-fetoprotein and Erythropoietin (EPO), etc.]; proteolipids (e.g., N-myristoylated, palmitoylated and prenylated proteins); and glycolipids (e.g., glycoglycerolipids, glycosphingolipids, glycophosphatidylinositols, etc.); (f) biologically derived homopolymers, such as polylactic and polyglycolic acids and poly-L-lysine; (g) nucleic acids (e.g., DNA, RNA, etc.); (h) hormones (e.g., anabolic steroids, sex hormones, insulin, angiotensin, etc.); (i) enzymes (types: oxidoreductases, transferases, hydrolases, lyases, isomerases, ligases; examples: trypsin, collegenases, matrix metallproteinases, etc.); (j) pharmaceuticals (e.g., beta blockers, vasodilators, vasoconstrictors, pain relievers, gene therapy, viral vectors, anti-inflammatories, etc.); (k) cell surface ligands and receptors (e.g., integrins, selectins, cadherins, etc.); and (1) cytoskeletal filaments and/or motor proteins (e.g., intermediate filaments, microtubules, actin filaments, dynein, kinesin, myosin, etc.).

In some embodiments, the MEA device cell culturing surface can be coated with materials that promote electroconductivity. In some embodiments, such material can be selected from the group consisting of charcoal, graphene, graphene oxide, reduced graphene oxide, nanotubes, titanium (Ti) and gold (Au), whereby electrical conductivity, or other physico-chemical property of the cultured cells can be amplified from the whole monolayer of cells present on the cell culturing surface. In some embodiments, the MEA device comprises electroactive polymer fibers that yield fibers that exhibit crystalline structures in polar form due to strong electromagnetic fields. Exemplary systems and methods for aligning the fibers are disclosed in US Application 2009/0108503, which is incorporated herein in its entirety by reference.

Depending on the base substrate employed, examples of surface layers on the MEA device that are electroconductive materials, including for example, but not limited to, a thin metallic or conducting surface deposit, e.g. graphene, graphene oxide and reduced graphene oxide (rGO), carbon nanotubes, tantalum, titanium, Ti—Al—V alloys, gold, chromium, metal oxides, semiconductor oxides, metal nitrides, semiconductor nitrides, polymers, biopolymers, or other alloys. In some embodiments, surface compositions can include, for example, graphene and graphene oxide, as well as tantalum, titanium, platinum or an oxide thereof. In some embodiments, the conductive surface layer can include modified structures of the above identified materials, including for example, carbon nanotube structures, metallic (e.g., gold, silver, and titanium) projections (e.g., pillars, posts, bumps, and ridges), and nanocavities.

### Uses of the MEA Devices

In some embodiments, the microelectrode arrays can be used to stimulate and record signals from cells cultured on the cell culturing surface. In some embodiments, a signal generator can be used to provide electrical input to the microelectrode arrays which are contact the cultured cells. In alternative embodiments, the microelectrode arrays can be used to record from the cells cultured on the cell culturing surface. A whole range of biopotentials and physico-chemical properties can be recorded from the cells cultured on the MEA device, e.g., but not limited to, functional parameters, including, but not limited to action potential duration (ADP), wave propagation, action potential frequency, beat frequency, action potential transmission, Vmax of the action potential, contraction force, peal to peak amplitude, end diastole to peak diastole rate and the like.

In some embodiments, the MEA device comprising electroconductive cells can be used in methods and assays for toxicity screening, e.g., to assess the effect of an agent on the biopotentials of the cultured cells, e.g., to identify any agent which adversely effects the biopotential of the cultured cells.

In some embodiments, the MEA device comprising electroconductive cells can be used in drug screening methods and assays, e.g., to assess the effect of potential therapeutic agents for the treatment of a disease or disorder. In some embodiments, for example, where the cultured cells are derived from a subject with a cardiovascular disease or disorder, or a subject having arrhythmia, the MEA comprising electroconductive cells can be used to screen for, and identify agents which restore, either partially or completely, the biopotential and electroconductive properties of the cells back to a more normal phenotype. In such embodiments, the cells can be human cells, which are originally derived from iPSC and differentiated into electroconductive cells, e.g., neuronal and/or muscle cells as described herein.

In some embodiments, the MEA device comprising electroconductive cells can be used for disease modeling, e.g., to see the effect of a particular transient electric signal on the phenotype of the electroconductive cells. In some embodiments, the phenotype can be the morphology or cell characteristics (e.g., expression of particular markers) or the chemo-electric properties of the cell.

In alternative embodiments, the MEA device can be used to assess the effect of different electrical signals on the development, differentiation, maturation, functionality and/or survival of electroconductive cells. For example, one can use the MEA device to provide electrical signals to the cells to induce them to differentiate along a particular lineage, e.g., to differentiate stem cells into an electroconductive cell type, e.g., neuronal and muscle cells, and subtypes thereof, including cardiomyocytes, skeletal myocytes and the like. Similarly, in some embodiments, one can use the MEA device to provide electrical signals to immature electroconductive cells to enhance their maturation to a more mature phenotype, e.g., by way of an example only, the MEA can be used to enhance the maturation of immature cardiomyocytes to more mature cardiomyocytes, e.g., with characteristics of mature adult cardiomyocytes found in vivo.

In some embodiments, the MEA device comprising electroconductive cells can be used in the identification of changing action potentials in muscle cells (e.g., cardiomyocytes), and thus can be used in the diagnosis of a cardiovascular disease or disorder, including, but not limited to arrhythmia in a subject. In some embodiments, where the electroconductive cells are skeletal muscle cells, the MEA device comprising skeletal muscle cells can aid the diagnosis of neuromuscular diseases and disorders, and/or myogenic disorders such as ALS, muscular dystrophy and the like. Similarly, the MEA device comprising electroconductive cells can be used in the identification of changing action potentials of neuronal cells, and thus can be used in the diagnosis of neurological and/or disorders and neurodegenerative disorders, e.g., including but not limited to, Parkinson's disease, Huntington's disease, ALS, Alzheimer's disease. In such embodiments, the electroconductive cells can be differentiated from cells obtained from the subject, e.g., electroconductive cells derived from iPSC originally obtained from the subject.

Electrocardiography depends on measurement Electromyography and electroencephalography function similarly in the diagnosis of neuromuscular and brain disorders, respectively.

In some embodiments, the MEA device can be part of a system, e.g., where the MEA device is connected to an electronic interface (e.g., signal generator and/or signal recorder), which can be optionally connected to a computer, where the computer instructs the electronic interface, whereby the computer directs the transmission of electric signals to and from the MEA device. In some embodiments, the MEA device of the system comprises electroconductive cells.

### BRIEF DESCRIPTION OF THE FIGURES

FIGS. 1A-1B show patterns on a shadow mask. FIG. 1A shows a microelectrode array (MEA) mask pattern on a shadow mask. Dark portions indicate opening in the shadow mask (nanogrooved PDMS layer is exposed to metallization) and clear portions are to be masked (no metallization). FIG. 1B depicts an embodiment where the shadow mask is placed on top of the nanogrooved PDMS layer and Cr/Au is deposited in a sputter using an optimized recipe. Clips are used to physically attach the shadow mask to the device. A spectral pattern is clearly seen on the pads, left and top, where nanopatterns reside.

FIGS. 2A-2B show MEA pattern on PDMS nanogrooves. FIG. 2A shows 2×3 MEA on a broken sample, MEA is made of Cr/Au sputtered directly on a nanogrooves layer using a shadow mask. FIG. 2B shows 2×4 MEA; the right edge of the nanogrooves layer is chopped. MEAs are formed at the center, having leads to the edge of the layer, connected to pads, allowing direct interface of biopotentials to the readout electronics.

FIGS. 3A-3C depict electron microscopy (SEM) of MEA on nanogrooves. FIG. 3A shows 2×3 MEA showing electrodes and leads. The electrodes are to contact cells to acquire biopotentials whereas electrical leads transfer the biopotentials to the interface electronics. FIG. 3B shows a magnified view of the edge of electrode. The Cr/Au electrode sits on top of the nanogrooves, demonstrating little degradation on nano-meter scale features of the PDMS nanogrooves. The edge of the electrode is also visually visible. FIG. 3C shows a magnified view of the nanogrooves covered by Cr/Au electrode.

FIGS. 4A-4B show electrical interface of the MEA on nanogrooves. FIG. 4A shows a socket having an array of electrical interconnects mates directly on top of the pads on the nanogrooves layer (no wires or wirebonds). This direct interface helps reducing parasitics associated with wires/wirebonds to acquire more accurate biopotentials. Wiring socket mounting on the MEA chip: number represents pin number of the socket (#1 is set to be ground) FIG. 4B shows a magnified view of pin number 45/46 (the pin number starts from bottom left corner of the socket and increases in counter-clockwise direction). Ag epoxy/paint is added to connect electrical leads to pads and socket pins. Some interconnects need to be manually connected using Ag epoxy/paint. These connections can be eliminated by using integrated fabrication technique.

FIGS. 5A-5B depict characterizing MEA. FIG. 5A shows electrodes are lumped and wired to a signal generator (input) and measure the signal from pins located at the perimeter of the socket. FIG. 5B shows a close-up of the area circled in FIG. 5A, showing a view of the ground connection.

FIG. 6 shows an electronic interface characterization setup: electrical signal from the MEA through socket pins goes through a series of signal conditioning blocks (i.e., amplifiers and filters), and then DAQ to be digitally sampled. The DAQ is connected to a PC having LabVIEW to visualize the signal.

FIG. 7 shows a temporal signal from the MEA using sine wave input from a signal generator (87.7 μV at 200 Hz), acquired from pin #7: (top) digitally filtered and (bottom) raw signal (without digital filter). The raw signal is modulated on top of low frequency components including 60 Hz noise. Digitally filtered signal still contains low frequency noise which needs to be removed by shielding or providing well-grounded source.

FIGS. 8A-8B depict frequency responses. FIG. 8A shows that with input: 58.5 μV @ 200 Hz Envelop (60 Hz noise) becomes clearer as input amplitude decreases. FIG. 8A shows that with Input: 87.7 μV @ 500 Hz Frequency response seems to be adequate, indicating the sampling rate is high enough and parasitics associated with wires and pads have little impact on the measurement.

FIGS. 9A-9B depict pin data. FIG. 9A shows data acquired at pin #8, input: 58.5 μV @ 100 Hz Pin #8 is located at the bottom (see slide 3) of the socket, right next to pin #7. The peak to peak amplitude is −400 mV. FIG. 9B shows data acquired at pin #19, input: 58.5 μV @ 100 Hz. Pin #19 is located at the right (see FIG. 45) of the socket, orthogonal to pin #7 & #8. The peak to peak amplitude is still ˜400 mV, which is similar to the one from pin #8. This result indicates the effective resistance and capacitance of two orthogonal electrical leads influence little on the measurement.

FIG. 10 shows data acquired at pin #33, input: 58.5 μV @ 100 Hz. Pin #33 is located at the top (see slide 3) of the socket, opposite side of pin #7 & #8. The peak to peak amplitude is −400 mV, similar to what we got from other pins.

FIGS. 11A-11B depict cylindrical culture chamber and MEA configuration. FIG. 11A shows a cylindrical culture chamber attached to the MEA on the nanogrooves layer. The chamber can be glued directly on the cell culturing layer using a silicone vacuum grease. The grease and cylinder can be auto-clavable and dry-heat sterilized prior to forming the culture if needed. FIG. 11B shows a 3×5 MEA array, showing the electrodes are exposed and the rest (field) is insulated by a thin layer of parylene (˜4 μm). In order to passivate electrical leads (otherwise multiple cells on the leads generate multiple biopotentials so that biopotentials can not be discernable) the cell culturing surface can be covered by parylene except the electrodes area.

FIGS. 12A-12C demonstrate the mechano-sensitivity of cell and tissue behaviors to nano-topographic variations in biomaterials. FIG. 12A depicts directed cell polarization along the grooves of different curvature. FIG. 12B depicts differential degree of primary cardiac cell protrusion into a 400-nm-wide groove (left) and an 800-nm-wide groove (right). FIG. 12C depicts fibroblast gradient response to variable ridge pattern arrays with graded spacing (left) and regularly spaced topographic pattern arrays (right).

FIG. 13 depicts an overall schematic illustration of an exemplary embodiment of the multi-well MEA (multi-electrode-array) described herein on nanogrooves, mimicking in-vivo environments: MEA, up to 128 channels, is integrated directly on nano-meter scale grooves (nanogrooves; width, pitch, and depth of 100s of nm) forming on a biocompatible flexible layer (i.e., PDMS (Polydimethylsiloxane) or PU (Polyurethane)). The nanogrooves provide in-vivo mimicking environments for cells to be monitored. Cells are cultured in a well (culture chamber), sitting on top of MEA. Biosignals (biopotentials and impedance) are collected at the electrodes of MEA. The electrodes cover over the nanogrooves following their contours to maintain integrity of the nanoscale cues. The biosignals are transferred to wires through vias located at the bottom of the PDMS/PU layer, and amplified, filtered, and channel multiplexer. Serialized biosignals from multi-well MEA are collected and digitized to be seen at PC interface. Both biosignals (biopotentials and impedance) can be simultaneously visualized using a time multiplexing method in pseudo real time, by taking advantage of fast electronics over relatively slow biosignals.

FIGS. 14A-14D demonstrate a nanotopographically-patterned cardiac tissue construct. FIG. 14A depicts SEM images of ex-vivo adult rat myocardium demonstrating well-aligned myocardium (middle) attached to parallel aligned matrix fibers (bottom). FIG. 14B depicts a photograph of a large-area (˜3.5 cm²) nanopatterned cell culturing surface (top) and its cross-sectional SEM image (bottom). FIG. 14C depicts the formation of confluent, neonatal rat ventricular myocytes on unpatterned and nanopatterned surfaces Immunofluorescent images of sarcomeric α-actinin and cell nuclei with pattern directionality indicated by the bi-directional arrow. FIG. 14D depicts action potential propagation across cell monolayers cultured on unpatterned and nanopatterned surfaces. Isochrones spaced at 5 ms intervals.

FIG. 15 demonstrates the sensitivity of properties of the cardiac constructs to the underlying nanostructured surfaces. Both (left) connexin-43 protein expression and (right) action potential longitudinal conduction velocity (LCV) are affected by nano-topography dimension.

FIG. 16 depicts SEMs of 2×3 MEA on nano-grooves PDMS: (left) low magnification (70×) showing electrodes and leads of 2×3 MEA. (middle) close-up view (1000×) of the edge of the electrode, showing Cr/Au thin film metal is covering the periodic nano-grooves well. (right) the magnified view (50,000×) of the nano-grooves covered by Cr/Au. The periodic and sharp features of nano-grooves are well maintained after the Cr/Au MEA formation.

FIGS. 17A-17D depict the formation of MEA on nanogrooves: FIG. 17A depicts an image of 3×5 MEA on nanogrooves: Leads are covered by a parylene-C film and electrodes are uncovered to expose to cells. FIG. 17B depicts a cylindrical culture chamber attached to the MEA on nano-grooves. FIGS. 17C-17D depict temporal signal acquisition of MEA on nano-grooves. An emulated source was used at the input from a signal generator: (FIG. 17C) 87.7 μV at 200 Hz and (FIG. 17D) 29.25 μV at 100 Hz. Signals look clear and frequency response is adequate. 60 Hz noise exists, which can be lowered by additional shielding wires and well-defined ground.

FIG. 18 depicts the fabrication sequence of a single-well MEA. Step (a): metallization on a glass slide, step (b): nanogrooves PDMS/PU transfer to the glass slide, step (c): formation of metal mask using 1^{st} shadow mask, step (d): dry etch of PDMS/PU and mask removal, step (e): electroplating vias, step (f): formation of electrodes array using 2^{nd} shadow mask, step (g): culture chamber attachment.

FIGS. 19A-19B depict the formation of confluent monolayer of NRVMs on the (FIG. 19A) unpatterned and (FIG. 19B) nanopatterned surfaces.

FIG. 20A depicts one example of a multi-well cell culture device integrated with large area nanotopographic surfaces for parallel screening of cellular response to variable surface topography and rigidity. FIG. 20B depicts SEM images of the fabricated patterns with different groove width.

FIG. 21 depicts a schematic of the manufacturing sequence of one embodiment of a multi-well MEA: step (a): two-step (thin-/thick-films) metallization on a glass slide: thin film is for wires and thick film is for pads, step (b): nanogrooves PDMS/PU transfer to the glass slide, step (c): vias/electrodes formation, step (d): culture chamber array made of PDMS, step (e): plasma-assisted bonding of the culture chamber array to the MEA device.

FIG. 22 depicts a schematic of signal detection uncertainty analysis: when signal line is orthogonal to the nanogrooves (direction 1) the RC time constant associated with the signal line is 3.84 (=1.96²) times larger than that of the line in parallel to the nanogrooves (direction 2). For single-well MEA, the acquisition of biosignals is irrelevant to this RC time constant variation as a function of nanogrooves. However, the signal acquisition in multi-well MEA, when time-multiplexing of biosignals is performed, suffers from the RC time constant variation. In order to eliminate the RC time constant variation, the wires are embedded between the PDMS/PU and glass slide (FIGS. 18 and 21).

FIG. 23 depicts a signal-chain block diagram of electronic interface to the multi-well (96 well) multi-channel (128 channels per well) MEA on nanogrooves: each electrode corresponds to a channel and one well contains up to 128 channels. Individual channels are processed through time multiplexing to convert the parallel channels to a serial signal. All channels are amplified and filtered before going through a multiplexer (MUX) for time multiplexing. The serialized signals from multiple wells are digitized (DAQ) in parallel and go through digital signal processor (DSP) to further refine the signals. Both biopotentials and impedance (Z) are through the same signal channels, except that impedance measurement requires external stimuli (AC voltage) provided at DAQ. DAQ generate stimulus voltage, which is demuxed (bottom) to provide analog AC voltage to individual channels (electrodes) to measure AC current to compute impedance (Z). The time division multiplexing allows pseudo-real-time acquisition of biosignals (bottom). Each MEA well is processed in parallel such that the DAQ retrieves signals simultaneously from the 96 wells.

FIG. 24 depicts a photograph of one embodiment of a MEA nanodevice as described herein. MEA nanodevice can be fabricated in different sizes, to ensure compatibility with arbitrary amplifier sockets, e.g, compatible with MCS 1060 amplifier or with Axion Biosciences.

FIGS. 25A-25E depict alternative layering of MEA nanodevice. FIGS. 25A-25C depict electrode-on-nano MEA nanodevice. FIG. 25A depicts a schematic showing the design principle of electrode-on-nano device. Electrodes are sputtered, or deposited in an alternative manner, on polymeric nanogrooves (PDMS/PUA/PEG and other UV curable polymers); FIG. 25B depicts a photograph of an embodiment of a 3×5 electrode-on-nano MEA nanodevice: leads are covered by a parylene-C film and electrodes are uncovered to expose to cells; FIGS. 25C-25E depict SEMs of 2×3 MEA on nano-grooves PDMS: FIG. 25C depicts low magnification (70×) showing electrodes and leads of 2×3 MEA; FIG. 25D depicts close-up view (1000×) of the edge of the electrode, showing Cr/Au thin film metal is covering the periodic nano-grooves well; FIG. 25E depicts the magnified view (50,000×) of the nano-grooves covered by Cr/Au. The periodic and sharp features of nano-grooves are well maintained after the Cr/Au MEA formation. An example of the electrode-on-nano MEA nanodevice with gold electrodes sputtered over PUA nanopatterns is depicted in FIG. 24.

FIGS. 26A-26B depict an exemplary embodiment of a Nano-on-electrode MEA nanodevice. FIG. 26A depicts a schematic showing spin coated polymeric nanogrooves created over a prefabricated MEA. FIG. 26B depicts an example of a nano-on-electrode MEA device with spin coated nanotextured area (bordered by dashed line) over a Multichannel 64 pin microarray with a customized culture chamber.

FIGS. 27A-27B demonstrate that cardiomyocytes form monolayers on MEA nanodevices readily, when seeded after coating with appropriate extracellular matrix molecules or synthetic peptides. Human pluripotent stem cell derived cardiomyocytes cultured on nano-on-electrode (FIG. 27A), and electrode-on-nano (FIG. 27B) MEA nanodevices form monolayers with cells aligned in the direction of nanogrooves. Arrow shows the direction of nanogrooves.

FIGS. 28A-28D demonstrate that the devices described herein do not appreciably change field potential of cultured cardiomyocytes. Spontaneously generated field potential profiles of neonatal rat ventricular myocytes cultured on MEAs with unpatterned (FIG. 28A), and nanotextured (FIG. 28B) polymer on electrodes. Spontaneously generated field potential profiles of human pluripotent stem cells derived cardiomyocytes cultured on MEAs with unpatterned (FIG. 28C), and nanotextured (FIG. 28D) polymer on electrodes.

FIGS. 29A-29D demonstrate that MEA nanodevices as described herein are more sensitive in detecting effect of drugs on cardiac activity on cells matured and anisotropically aligned over the nanotextured platform. (FIGS. 29A-29B) Addition of 0.1 μM cisapride, a hERG channel inhibitor, causes detectable aberration in field potential profiles of human pluripotent stem cell derived cardiomyocytes cultured over non-textured (unpatterned) MEAs. Shown here are representative field potential profiles from non-textured MEAs before (FIG. 29A), and after addition of 0.1 μM cisapride (FIG. 29B). (FIGS. 29C-29D) MEA nanodevice shows a detectable aberration effect at a much lower dosage of 0.02 μM cisapride in field potential profiles of human pluripotent stem cell derived cardiomyocytes cultured and matured over MEA nanodevice (textured). Shown here are representative field potential profiles from MEA nanodevice before (FIG. 29C), and after addition of 0.02 μM cisapride (FIG. 29D).

FIGS. 30A-30B demonstrate that a MEA nanodevice as described herein can accurately detect drug that are known hERG inhibitor, but is recognized as a false positive. Verapamil, a known hERG inhibitor, causes dose dependent aberration in field potential profiles of human pluripotent stem cell derived cardiomyocytes cultured on non-textured MEAs (FIG. 30A), but does not cause detectable aberration in MEA nanodevice (FIG. 30B). Above, field potentials shown after 2 minutes of perfusion with verapamil at shown dosages.

FIGS. 31A-31B demonstrate that MEA nanodevices as described herein are more sensitive in detecting effect of drugs on cardiac activity on cells matured and anisotropically aligned over the nanotextured platform. (FIG. 31A) Addition of 1 μM Calcium channel inhibitor causes detectable aberration in field potential profiles of human pluripotent stem cell derived cardiomyocytes cultured over non-textured (unpatterned) MEAs. Shown here are representative field potential profiles from non-textured MEAs after addition of 1 μM drug. (FIG. 31B) MEA nanodevice shows a detectable aberration effect at a much lower dosage of 0.02 μM cisapride in field potential profiles of human pluripotent stem cell derived cardiomyocytes cultured and matured over MEA nanodevice (textured). Shown here are representative field potential profiles from MEA nanodevice addition of drugs.

### DETAILED DESCRIPTION

The present invention is directed to microelectrode array (MEA) based devices and methods for using these devices including, for example, in the study of cells and tissue. In accordance with the MEA devices according to the invention, cells can be cultured more naturally, enabling the cultured cells and tissue to be studied in-vitro, but in a more natural context. In accordance with some embodiments of the invention, the MEA devices include a cell culture surface upon which the cells and tissue to be studied can be cultured. The cell culture surface can include micrometer and nanometer sized features that encourage the cells and tissue to culture in configurations that more closely model the way the cells and tissues would develop in the body, such as when using an extracellular matrix (ECM).

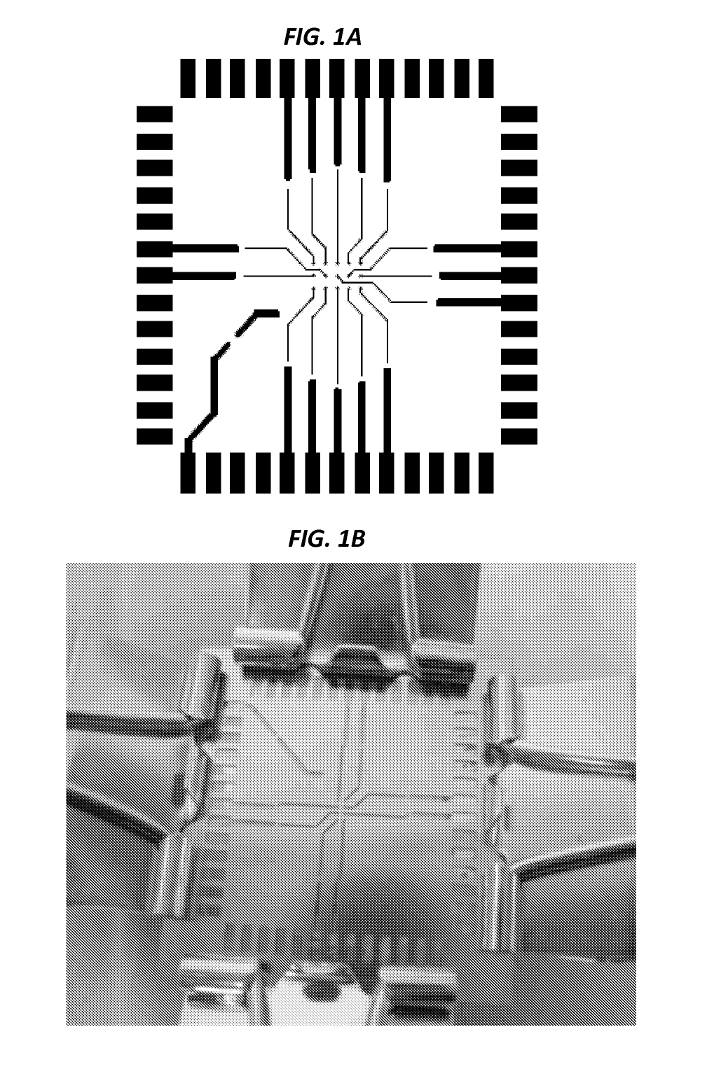

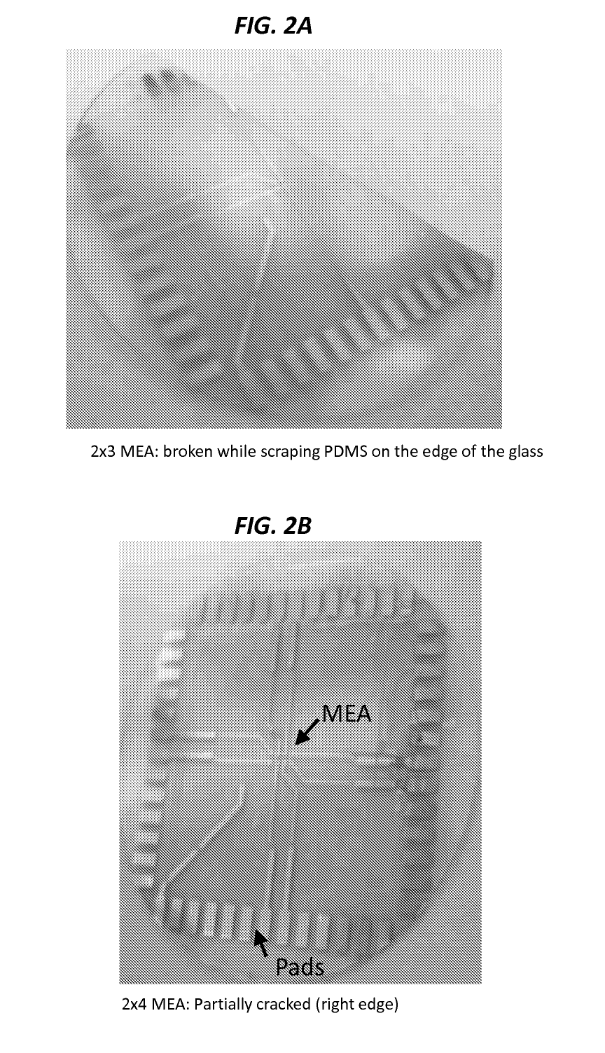

FIGS. 1A and 1B show the process for applying metallization to the MEA that forms the electrodes and the connecting leads that provide the electrical connection with the external signal recording/monitoring and signal generating components. FIG. 1A shows an MEA mask pattern on a shadow mask according to some embodiments of the invention. In this embodiment, the dark portions indicate the openings in the shadow mask where the biocompatible cell culturing layer is exposed to metallization and the clear portions are to be masked (no metallization). In accordance with some embodiments of the invention, the shadow mask is placed on top of the nanogrooves PDMS cell culturing layer and Cr/Au is deposited using a sputter process. Clips or clamps can be used to physically attach the shadow mask to the biocompatible cell culturing layer during the metallization process. In accordance with some embodiments, the metallization can be applied over the biocompatible cell culturing layer after the nanopatterning (e.g., nanogrooves) has been formed in the cell culturing layer. A spectral pattern is clearly seen on the pads, left and top, where nanopatterns reside. In accordance with some embodiments, the metallization can be applied to a base substrate and a biocompatible (e.g. polymer) layer that includes the nanogrooves (or is later processed to include the nanogrooves) can be applied over the metallization layer on the base substrate. FIGS. 2A and 2B show examples of the MEA electrode patterns formed on a PDMS cell culturing layer that includes nanogrooves. Each electrode can include a lead (e.g., a wire) that provides electrical connection between the electrode and a pad that is formed on the peripheral edge of the device. The pad facilitates electrical connection to external signal detection/monitoring/recording systems and signal generation systems.

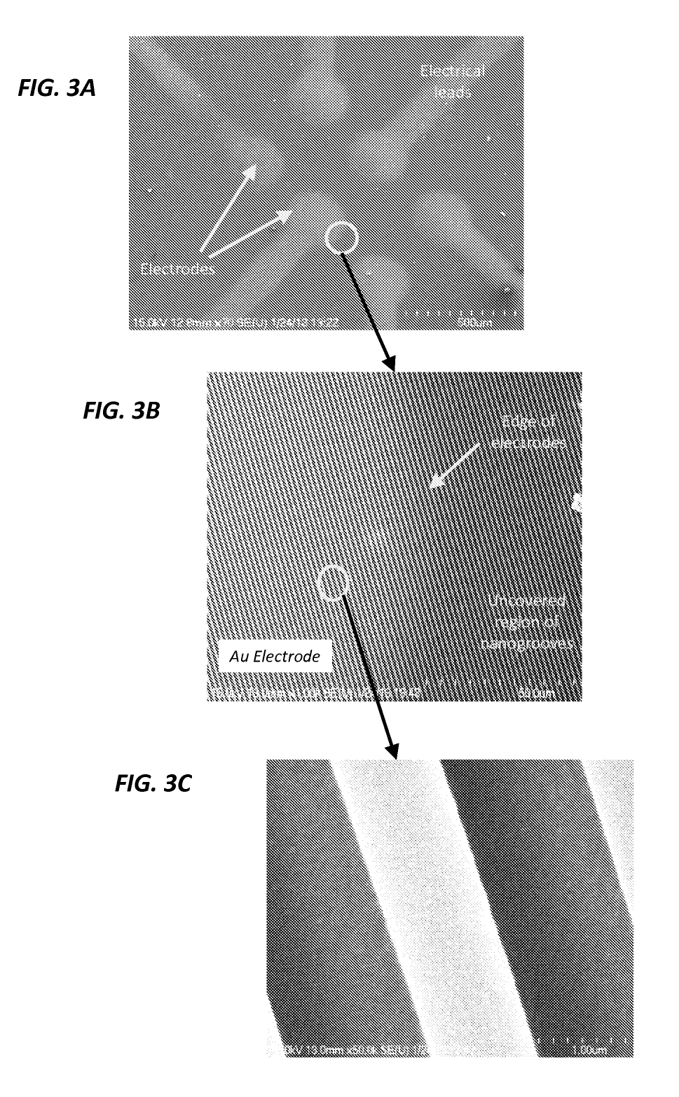

FIGS. 3A, 3B, and 3C show scanning electron microscope images of the MEA formed on the nanogrooves of the biocompatible cell culturing layer. FIG. 3A shows an example of Cr/Au electrodes in a 2 by 3 array pattern. FIG. 3B shows an enlargement of the circled portion showing a portion of an electrode formed on the nanogroove. FIG. 3C shows an enlargement of the Cr/Au electrode formed over the nanogrooves wherein the Cr/Au covers the tops of the ridges the separate the nanogrooves as well as the side wall surfaces and bottom surface of the nanogroove. A layer of insulating material can be applied over the leads extending from the electrodes to prevent or reduce noise and unwanted signals from being introduced into the circuit.

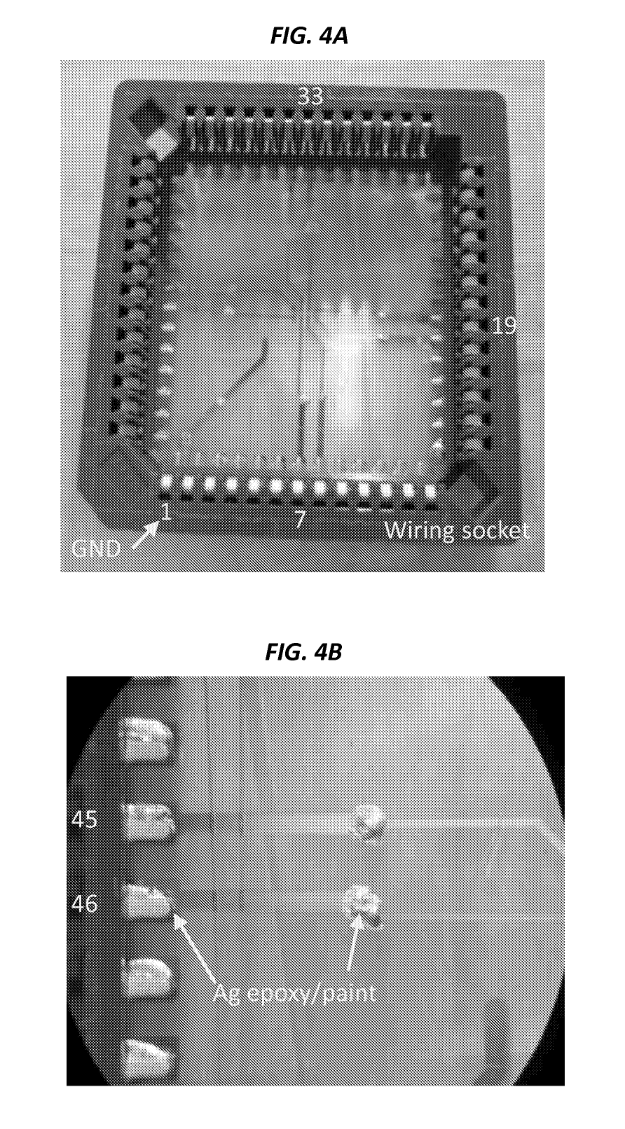

In accordance with some embodiments of the invention, the MEA devices can be formed in configurations compatible with standard integrated circuit devices such that the MEA devices can be removably inserted into integrated circuit type sockets in order to provide electrical connections between the MEA devices and external signal sensing and signal generating components. FIGS. 4A and 4B show an MEA device mounted in a wiring socket to facilitate external electrical connection. In accordance with some embodiments of the invention, various electrical interconnects can be used to connect the electrodes in the array with a peripheral pad. As shown in FIG. 4B, Ag epoxy paint can be used to connect the leads to the pads without the use of heat which can damage a polymer cell culturing layer. Other metallization processes can also be used, for example, a second mask process that includes sputter deposition can be used to apply additional layers. Well known etching (e.g., chemical etching, laser etching, dry etching, reactive ion etching) techniques can be used to remove portions of the biocompatible cell culturing layer for electrical connections as well as to form the micrometer and nanometer grooves.

In accordance with some embodiments of the invention, the MEA device can be formed by a process that includes applying layers sequentially to build up cell culturing surface. The pads, leads, and optionally, electrodes can be applied to a base substrate using a first mask and sputter deposition of conducting material. A cell culturing layer of biocompatible polymer or dielectric material can be applied over the surface and the layer can include the micro/nano patterning formed on the surface or the micro/nano patterning can be formed in a subsequent step. Optionally, holes and conducting vias can be formed in the cell culturing layer to provide electrical connection to electrodes that can be applied to the micro/nano patterned surface. The optional surface electrodes can be applied to the surface using a second mask and a sputter deposition process. Other well-known thin and thick film metal deposition processes can be used to apply the leads and the pads to the biocompatible cell culturing layer and/or the base substrate material.

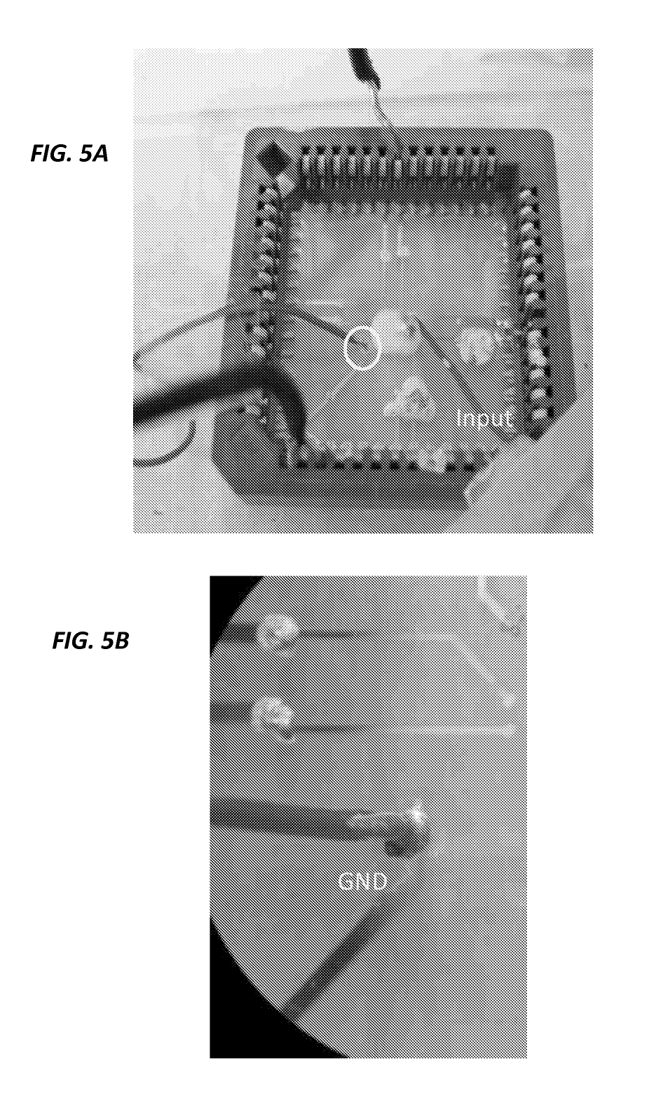

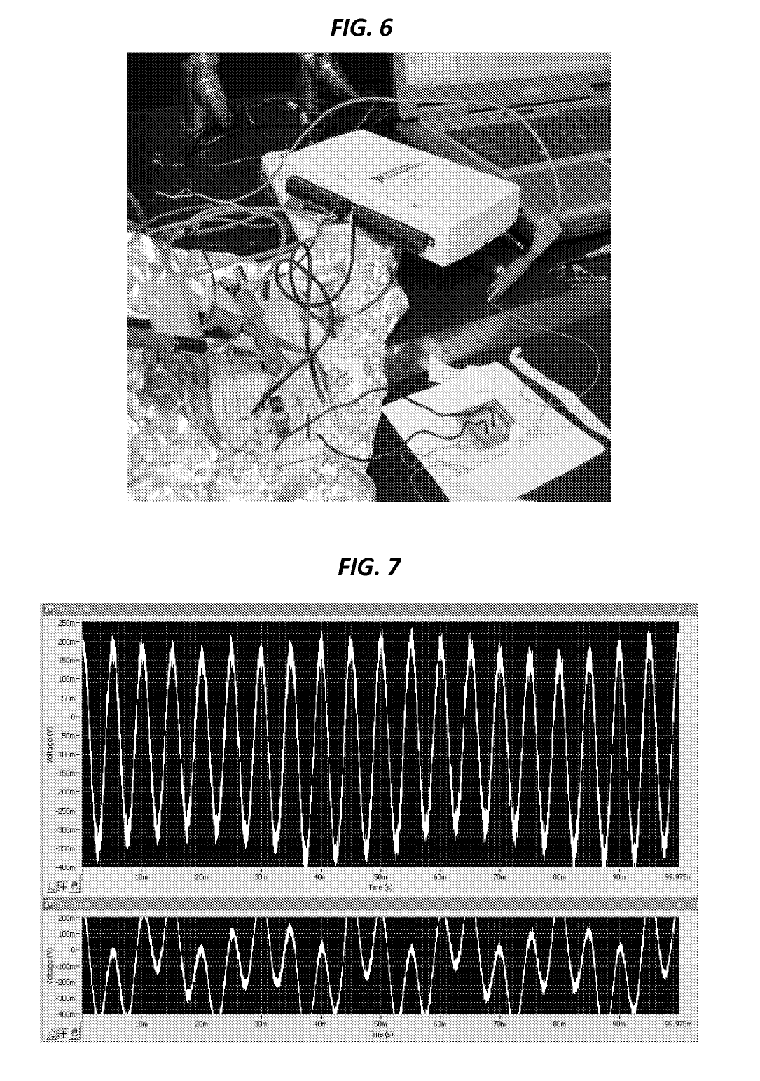

As shown in FIGS. 5A-5B and 6, the electrodes can be electrically connected to one or more signal generating devices and one or more signal processing devices. The signal generating devices can include electronic devices that produce electrical potentials intended to be sent to the cells and tissues cultured on the surface of the MEA to study the cell/tissue response. The signal processing devices include devices that receive signals from the cells and tissue and attempt to characterize the signals. The signal processing devices can include signal conditioning blocks such as amplifiers and filters, and data acquisition (DAQ) devices that digitally sample and characterize the signal. The signal processing devices can also include a storage device for storing the signals produced by the cells/tissue. In accordance with some embodiments of the invention, the signal processing device can include a personal computer (PC) having one or more processors and associated memories, equipped with an interface board to send and/or receive signals to more than one electrode simultaneously. The PC can include software, such as LabVIEW software, to visual the signals.

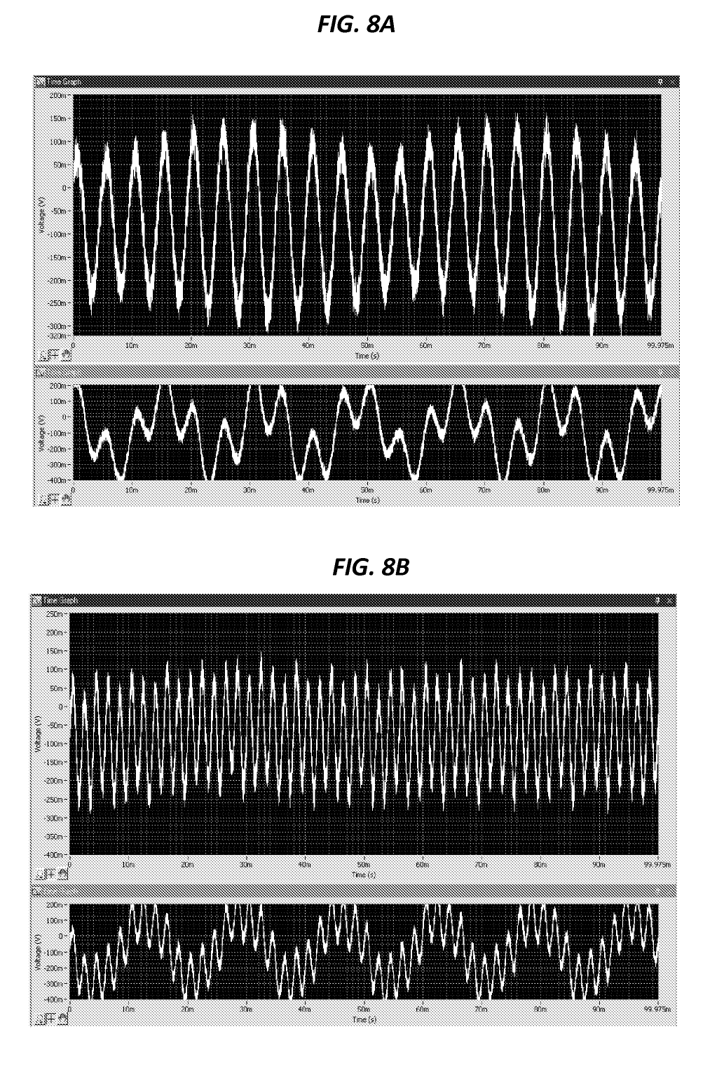

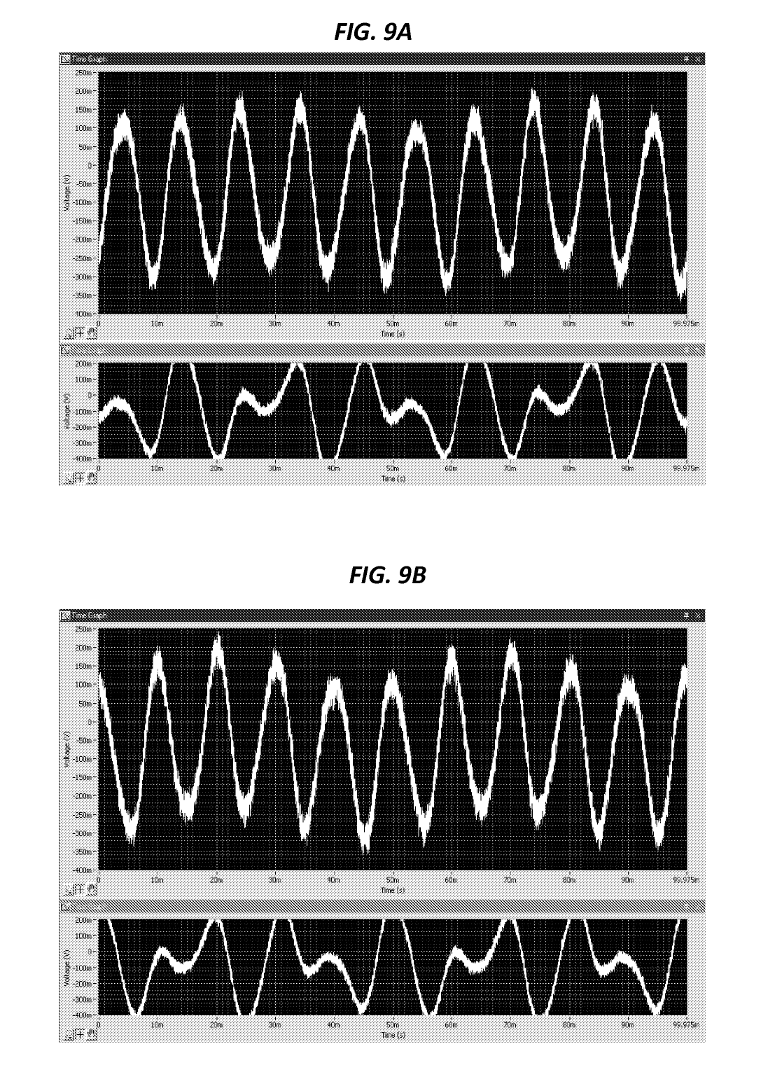

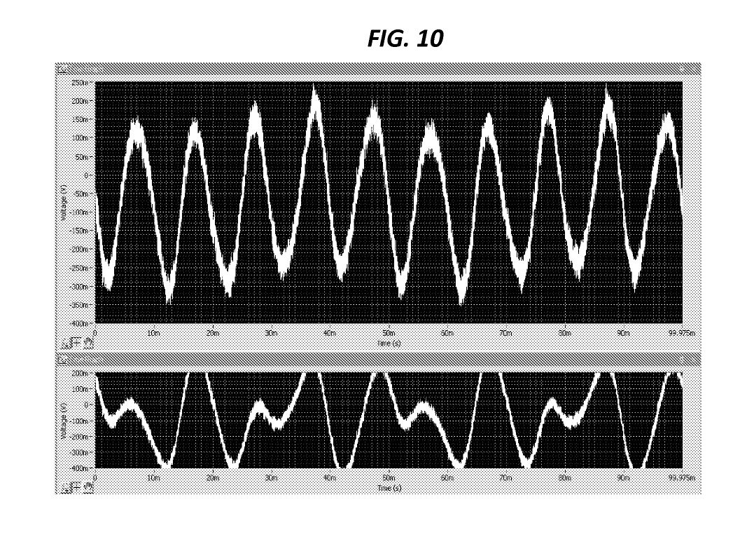

FIGS. 7, 8A-8B, 9A-9B, and 10 show images of the signals captured from various electrodes of the device shown in FIGS. 4A and 4B. The signals are captured from pins 7, 8, 19 and 33 which connect to the adjacent electrodes of the MEA device. The signals were sampled at sampling rates varying from 100 Hz to 500 Hz and demonstrate that the location or orientation of the lead have little influence on the signal measurement.

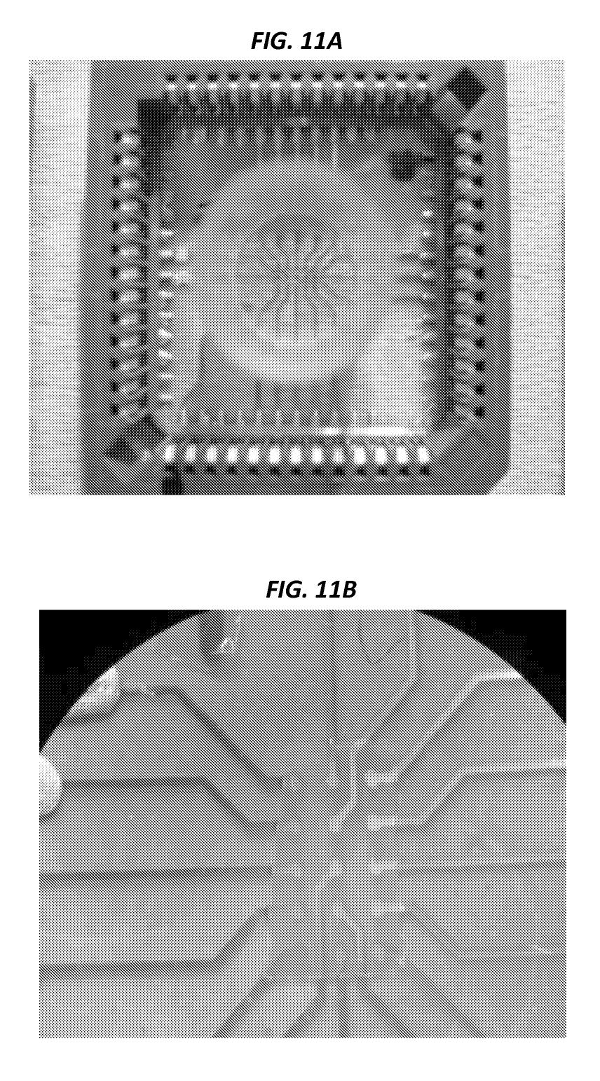

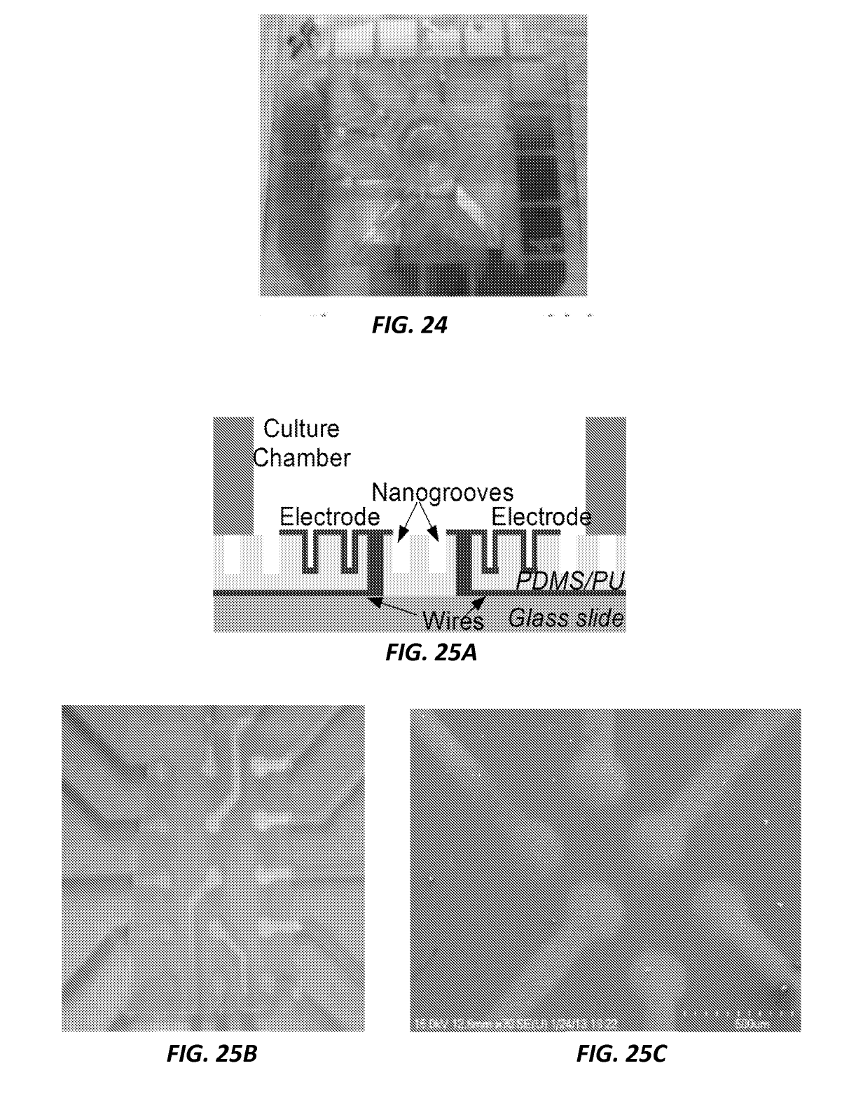

In accordance with the various embodiments of the invention, the MEA preferably includes a chamber that maintains cell/tissue viability by immersion in media. The chamber can be any structure that maintains the cells/tissue in the media. In accordance with some embodiments of the invention, the MEA can include chamber walls that encircle the MEA and encourage cells to culture over some of the electrodes in the array. The chamber can define any shape, from circular (as shown in FIGS. 11A and 24) or oval to regular and irregular polygons. In accordance with some embodiments, the shape of the chamber can be defined by the intended cell and tissue development or to encourage a predefined cell/tissue organization.

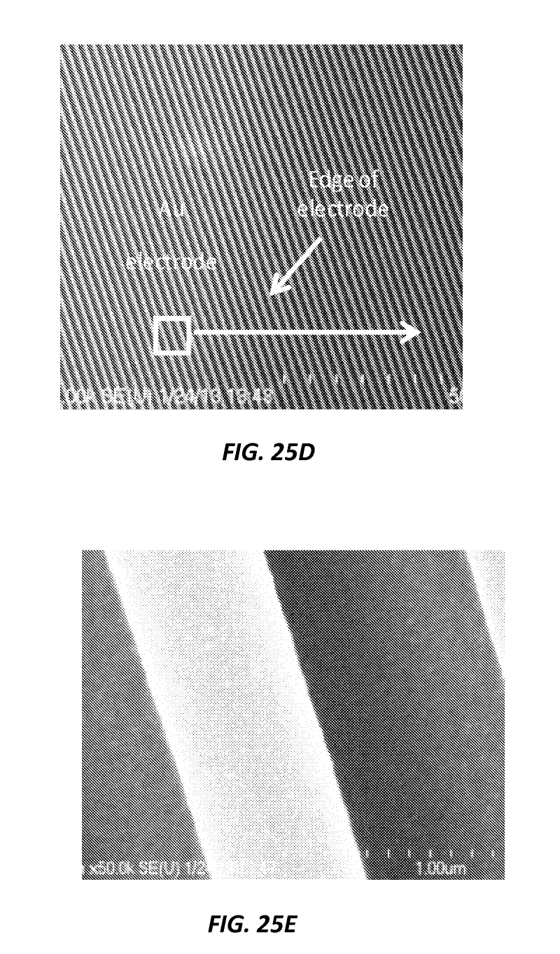

In accordance with some embodiments of the invention, where the leads of electrode are formed on the top of the cell culturing layer, it can be desirable to insulate the leads so they do not pickup signals from other cells that can interfere with signals received from cells in contact with the electrode. In accordance with some embodiments, the leads can be passivated by covering them with a thin layer of a biocompatible insulating material, such as parylene, shown in FIGS. 11B, 25B. The insulating material can be on the order of 1.0-5.0 μm thick.

In accordance with some embodiments of the invention, an MEA having a cell culturing surface can be modified according to the present invention. The MEA can include a plurality of electrodes arranged in any configuration on a cell culturing surface, the electrodes being connected by wires or leads to pad located at or adjacent to a peripheral portion of the device. In accordance with some embodiments of the invention, a biocompatible layer can be applied to the cell culturing surface of the MEA as shown in FIGS. 26A and 26B. The biocompatible layer in accordance with some embodiments of the invention can be any biocompatible material, for example, a flexible polymer such as Polydimethylsiloxane (PDMS) or Polyurethane (PU). In accordance with some embodiments of the invention, the flexibility and/or hardness of the biocompatible layer can be selected to encourage a more physiological response of cells arranged upon it. For example, to the extent that muscle cells in vivo can normally be arranged upon an extracellular matrix substrate that is naturally flexible, and gives when the cells contract, it is contemplated that the use of a flexible biocompatible layer can permit cells arranged thereupon to behave in a manner that better approximates muscle tissue in vivo. In some embodiments, such an arrangement can contribute to more accurate prediction of, e.g., drug effects on cardiac muscle in vivo using the multielectrode array constructs described herein. In accordance with some embodiments of the invention, the biocompatible layer can be a rigid, semi-rigid, or flexible material selected to approximate, e.g., bone, cartillage or an extracellular matrix to more closely approximate the hardness, flexibility and texture of the natural material on which the cells naturally develop.

In accordance with some embodiments of the invention, the MEA can be integrated with a flexible biocompatible layer (e.g., PDMS or PU), that possesses nanometer (or micrometer) scale features, such as grooves and/or ridges. In accordance with some embodiments of the invention, the nanometer grooves (or “nanogrooves”) can be in the range from 100 nm to 1000 nm in width, pitch, and/or depth, and can provide mechanical cues to the cells that are cultured on top of the nanogrooves. The nanogrooves and other nanometer scale features can be formed by well-known additive or subtractive fabrication processes, including etching (e.g., laser etching, chemical etching, dry etching and/or reactive ion etching) and stamping. The nanogrooves can be formed by capillary force lithography-based nanopatterning.

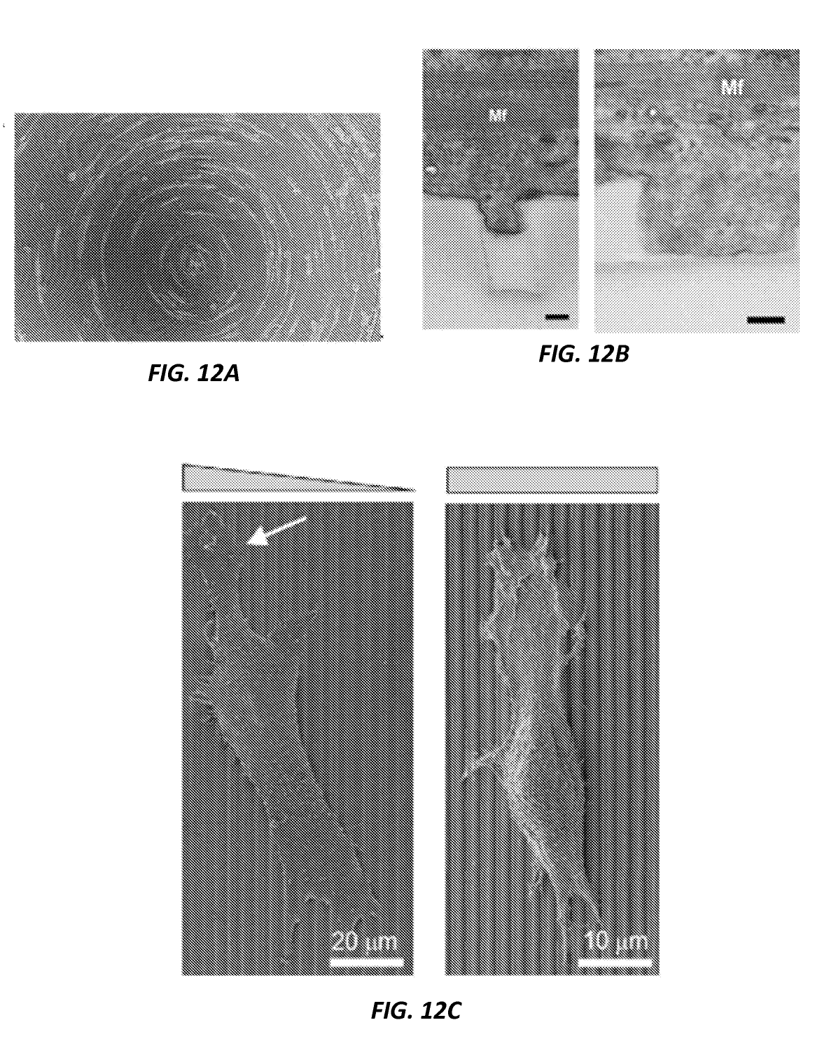

In accordance with the various embodiments of the invention, the nanometer and micrometer scale features can be configured and arranged on the cell culturing surface to encourage organization of cells and tissue during culturing and to test cell and tissue function. In accordance with some embodiments of the invention, the width, depth and/or pitch (e.g., spacing between nanogrooves) of the nanogrooves can be substantially uniform over the length of each nanogroove. In accordance with some embodiments of the invention, the width, depth and/or pitch (e.g., spacing between nanogrooves) of the nanogrooves can be substantially non-uniform (e.g., irregular) over the length of each nanogroove. In accordance with some embodiments of the invention, two or more of the nanogrooves can extend substantially parallel over at least a portion of the cell culturing surface. In accordance with some embodiments of the invention, the distance between two or more of the nanogrooves can vary over at least a portion of the cell culturing surface and can intersect one or more times. In accordance with some embodiments of the invention, one or more of the nanogrooves can be curved and the curve can be regular (e.g., having a constant radius of curvature) or irregular (e.g., having different radii of curvature over the length of the nanogroove). In accordance with embodiments of the invention, the nanogrooves can be formed concentric circles such as shown in FIG. 12A. In accordance with some embodiments, the width and depth of the grooves can be different resulting in a different amount of cell protrusion into the nanogroove. For example, FIG. 12B shows primary cardiac cell protrusion into a 400-nm-wide groove (left) and an 800-nm-wide groove (right). In accordance with some embodiments, the spacing between nanogrooves and/or the width of each nanogroove can vary over the cell culturing surface. For example, FIG. 12C shows of a Fibroblast response to variable ridge pattern arrays with graded spacing between nanogrooves (e.g., increasing left to right) and FIG. 12D shows a Fibroblast response to regularly spaced topographic nanogroove pattern arrays. Similarly, the depth of the nanogrooves can vary over the length of the groove and/or be different from one nanogroove to an adjacent nanogroove.

Further, the MEA devices according to the invention can be configured with compound configurations of nanogrooves and other nanometer sized features that can include different features in different regions or areas of the cell culturing surface. For example, one or more straight and uniform nanogrooves can extend along one area and an adjacent area can include a curved, a spiral and/or an irregularly configured portion.

According to some embodiments of the invention, the array of electrodes can be positioned or arranged in a predefined configuration with respect to the nanofeatures applied to the cell culturing surface. For example, the nanofeatures (e.g., nanogrooves or ridges) can be configured to cause the cells and/or tissue to align along a first axis and the array of electrodes can be positioned to contact the cells and/or tissue at predefined locations with respect to the axis. In another example, the cell culturing surface can include a region having a different or an irregular configuration of nanofeatures and the electrodes can be strategically placed with respect to those nanofeatures.

As used, the term “irregular” when used in reference to grooves or nanogrooves refers to configurations other than straight, uniform, and parallel sets of grooves or nanogrooves. In some embodiments, irregular grooves (or nanogrooves) are those with increased anisotropy relative to straight, parallel, and uniform grooves (or nanogrooves). In some embodiments, the irregular grooves (or nanogrooves) can comprise curved grooves, intersection grooves, spiral patterned grooves, semi-randomly configured grooves, and/or randomly configured grooves. In some embodiments, a portion of a cell culture surface comprises irregular grooves (or nanogrooves) while another portion comprises regular grooves (or nanogrooves) (e.g. straight, uniform, and parallel).

These irregular nanogrooves can permit the culture and/or study of, e.g. cardiomyocytes with arrhythmias and/or cardiomyocytes which are not properly aligned. Misaligned cardiomyocytes can be found in the cardiac tissue of subjects who have experienced a myocardial infarction, the cardiac tissue of subject with congenital heart defects, and/or in scar tissue of the heart. Such devices can be used, as described herein, to, by the way of non-limiting example, screen for anti-arrhythmic agents or agents to treat cardiac scar tissue and/or heart diseases.

In one aspect, described herein is a cell culture system comprising a multi-electrode array as described herein and an electroconductive cell present on the cell culture surface. In some embodiments, the electroconductive cell can be in contact with at least one electrode. In some embodiments, the electroconductive cell can be in close enough proximity to at least one electrode for the transmission of electrical signals between the cell and the electrodes, e.g. when arrays with electrodes located beneath the cell culturing surface and wherein the electrodes do not form a portion of the cell culturing surface are utilized.

In some embodiments, the cell culture system can comprise a plurality of electroconductive cells. In some embodiments, the plurality of cells can form a monolayer of cells. In some embodiments, the plurality of cells can form a tissue, e.g. a muscle tissue or nerve tissue. In some embodiments, the cell culture system can further comprise non-electroconductive cells, e.g., fat cells or epithelial cells.

As used herein, “electroconductive cell” refers to a cell being able to conduct, generate, and/or responds to an electrical signal. Non-limiting examples of electroconductive cells can include neurons and myocytes (muscle cells). Electroconductive cells can include both naturally-occurring electroconductive cells (e.g., a muscle cell or neuron) or cells that have been engineered, e.g., genetically modified or transfected to exhibit electroconductive activity. By way of non-limiting example, a cell engineered to express at least one voltage-gated ion channel can be an engineered electroconductive cell. One of skill in the art is familiar with methods for engineering cells, which can include, but are not limited to, genetic modification, homologous recombination, transient expression, and protein transfection and can be accomplished with one or more various vectors, e.g., plasmids, naked DNA, or viral vectors.

In some embodiments, the electroconductive cells can be muscle cells. In some embodiments, the muscle cells can be cardiomyocytes. In some embodiments, the muscle cells can be cardiac pacemaker cells. In some embodiments, the muscle cells can be smooth muscle cells. In some embodiments, the muscle cells can be skeletal muscle cells.

In some embodiments, the electroconductive cells can be neuronal cells.

In some embodiments, a cell culture system described herein can comprise a plurality of types of electroconductive cells.

In one aspect, described herein is a cell culture system comprising a multi-electrode array as described herein and a progenitor of an electroconductive cell present on the cell culture surface. In some embodiments, the progenitor cell can be a cardiac progenitor cell, a myogenically committed cell, a neuron progenitor cell, a stem cell, an embryonic stem cell, an iPS cell, an adult stem cell. In some embodiments, the cell culture system can further comprise electroconductive cells and/or non-electroconductive cells.

In some embodiments, the cells comprised by a cell culture system described herein, e.g. electroconductive cells can comprise cells obtained from or descended from cells obtained from a subject with a cardiac or neuronal disease. In some embodiments, the cardiac or neuronal disease can be a disease characterized or caused by aberrant electroconductive activity. Non-limiting examples of such cardiac and neuronal diseases can include arrhythmia, long QT syndrome, heart block, atrial fibrillation, bradycardia, tachycardia, ventricular fibrillation, Adams-Stokes disease, atrial flutter, Wolff-Parkinson White syndrome, peripheral neuropathies, Charcot-Marie-Tooth disease, and the like. In some embodiments, the cells can comprise a genetic mutation known to be associated with or to cause a cardiac or neuronal disease. In some embodiments, the cells can comprise a genetic background known to be associated with or to cause a cardiac or neuronal disease. In some embodiments, the cells can be obtained from, or descended from cells obtained from as ethnic background known to be associated with a cardiac or neuronal disease.

In some embodiments, the electroconductive cells can be electroconductive cells modified to comprise a mutation associated with a cardiac disease, e.g., engineered to comprise such a mutation. By way of non-limiting example, Charcot-Marie-Tooth disease is associated with certain mutations in, e.g., PMP22, MPZ, LITAF, EGR2, NEFL, KIF1B, MFN2, RAB7A, LMNA, MED25, TRPV4, GARS, HSPB1, GDAP1, DYNC1H1, LRSAM1, MTMR2, and SBF2, among others and long QT syndrome has been linked to mutations in KCNQ1, KCNH2, hERG, MiRP1, SCN5A, ankyrin B, KCNE2, KCNJ2, CACNA1C, Caveolin 3, SCN4B, AKAP9, SNTA1, and GIRK4. One of skill in the art is aware of further examples.

In one aspect, described herein is an assay for measuring the effect of an agent on electroconductive cells, the assay comprising contacting a cell culture system as described herein with the agent and measuring the growth, viability, or activity of the electroconductive cells. The agent can be a drug or drug candidate and the assay can assess the safety of the drug with regards to electroconductive cells. In some embodiments, the agent can be a drug or drug candidate for a cardiovascular, muscular, or neuronal disease or disorder.

The term “agent” refers to any entity which is normally not present or not present at the levels being administered to a cell, tissue, organ or subject. Agent can be selected from a group comprising: chemicals; small molecules; nucleic acid sequences; nucleic acid analogues; proteins; peptides; peptidomimetics; peptide derivatives; peptide analogs; aptamers; antibodies; intrabodies; biological macromolecules; or functional fragments thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. In some embodiments agents can be extracts made from biological materials such as bacteria, plants, fungi, or animal cells or tissues. In some embodiments, agents can be naturally occurring or synthetic compositions or functional fragments thereof. Agents can be known to have a desired activity and/or property, or can be selected from a library of diverse compounds.

Generally, agents can be tested at any concentration that can modulate the activity, growth, and/or viability of an electroconductive cell. In some embodiments, agents are tested at concentration in the range of about 0.1 nM to about 1000 mM. In one embodiment, the compound is tested in the range of about 0.1 μM to about 20 μM, about 0.1 μM to about 10 μM, or about 0.1 μM to about 5 μM. Depending upon the particular embodiment being practiced, the candidate or test agents can be provided free in solution. Additionally, for the methods described herein, test agents can be screened individually, or in groups. Group screening is particularly useful where hit rates for effective test agents are expected to be low such that one would not expect more than one positive result for a given group.

Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule (Woburn, Mass.), Invitrogen (Carlsbad, Calif.), Ryan Scientific (Mt. Pleasant, S.C.), and Enzo Life Sciences (Farmingdale, N.Y.). These libraries can be screened for the ability of members to modulate the activity, growth, and/or viability of electroconductive cells using e.g. methods described herein.

In some embodiments, the agents can be naturally occurring proteins or their fragments. Such agents can be obtained from a natural source, e.g., a cell or tissue lysate. Libraries of polypeptide agents can also be prepared, e.g., from a cDNA library commercially available or generated with routine methods. The agents can also be peptides, e.g., peptides of from about 5 to about 30 amino acids, with from about 5 to about 20 amino acids being preferred and from about 7 to about 15 being particularly preferred. The peptides can be digests of naturally occurring proteins, random peptides, or “biased” random peptides. Peptide libraries, e.g. combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

The agent can function directly in the form in which it is administered. Alternatively, the agent can be modified or utilized intracellularly to produce a form that modulates the desired activity, e.g. introduction of a nucleic acid sequence into a cell and its transcription resulting in the production of an activity within the cell.

In some embodiments, the agent that is screened and identified to modulate the activity, growth, and/or viability of an electroconductive cell to the methods described herein by at least 5%, preferably at least 10%, 20%, 30%, 40%, 50%, 50%, 70%, 80%, 90% relative to an untreated control. A level which is higher or lower than a reference level (e.g. the level in the absence of the agent) can be a level which is statistically significantly different than the reference level. In some embodiments, a level that is lower than a reference level can be 90% or less of the reference level, e.g. 90% or less, 80% or less, 70% or less, 60% or less, 50% or less, 25% or less, or 10% or less of the reference level. In some embodiments, a level that is higher than a reference level can be 1.5× or more of the reference level, e.g. 1.5× or more, 2× or more, 3× or more, 5× or more, or 10× or more of the reference level. The reference level can be the level in the absence of the agent, e.g. the level in a parallel, untreated cell culture well (e.g. of the same multi-well microelectrode array), the level in the cell and/or well prior to contact with the agent, and/or a level in a population of cells not contacted with the agent, e.g. a pre-determined level.

In some embodiments, the methods and assays described herein can relate to measuring or determining cell viability, e.g. after contacting electroconductive cells with an agent. “Measuring cell viability” refers to measuring or detecting any aspect of cell metabolism, growth, structure, and/or propagation which is indicative of either a healthy, viable cell or a dead and/or nonviable cell. Colorimetric, luminescent, radiometric, and/or fluorometric assays known in the art can be used. In some embodiments, determining cell viability can comprise manual counting of cells using a hemacytometer. In some embodiments, determining cell viability can comprise the use of a live-dead cell stain, e.g. a stain which will stain either a live cell or a dead cell.

Colorimetric techniques for determining cell viability include, by way of non-limiting example, Trypan Blue exclusion. In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in cell lysosomes; viable cells can be determined with a light microscope by quantitating numbers of Neutral Red stained cells.

Fluorometric techniques for determining cell viability include, by way of non-limiting example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochromelabeled antibody. The fluorescent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for determining cell proliferation include, by way of non-limiting example, [3H]-Thymidine, which is incorporated into newly synthesized DNA of living cells and frequently used to determine proliferation of cells. Chromium (51Cr)-release from dead cells can be quantitated by scintillation counting in order to quantitate cell viability

Luminescent techniques for determining cell viability include, by way of non-limiting example, the CellTiter-Glo luminescent cell viability assay (Promega Madison Wis.). This technique quantifies the amount of ATP present to determine the number of viable cells.

Kits for determining cell viability are commercially available, e.g. the MUTLITOX-FLOUR™ Multiplex Cytotoxicity Assay (Cat. No. G9200; Promega, Inc.; Madison, Wis.). In some embodiments, the means of determining cell viability can comprise a high-throughput method, e.g. live-dead cell stains can be detected using a fluorescence-capable multiplate reader. In some embodiments, imaging analysis can be performed via automated image acquisition and analysis.

Measurements of cell growth can include, e.g., measurements of changes in cell volume and/or size and/or measurement of cell proliferation. Changes in cell size can be measured, e.g. using imaging analysis software. Cell proliferation can be measured using, e.g., an MTS assay commercially available from a variety of companies including RnD Systems, and Promega, among others.

In some embodiments, the activity of the electroconductive cells can be an activity selected from the group consisting of field potential; field potential profiles; field potential duration; QT interval length, conduction velocity; electrophysical profile; spontaneous beating rate; reentrant wave patterns; and impedance.

In some embodiments, the assay can further comprise inducing arrhythmia in the cells and the measuring the ability of the agent to restore normal field potential properties. In some embodiments, the activity of the agent as an anti-arrhythmia agent is measured. Arrhythmia can be induced in the cells comprised by a cell culture system described herein by a number of methods. In some embodiments, arrhythmia can be induced by contacting the cells with an arrhythmia-inducing agent. Such agents are known in the art and can include, by way of non-limiting example, epinephrine or norepinephrine. In some embodiments, arrhythmia can be induced by providing irregular electrical stimulation to the cells via the multi-electrode array, e.g. an irregular pattern of electrical impulses, varying in magnitude and/or frequency. In some embodiments, arrhythmia can be induced by culturing the cells on a multi-electrode array having irregular nanogrooves. In some embodiments, arrhythmia can be induced by culturing the cells on a multi-electrode array in which a portion of the cell culture surface comprises irregular nanogrooves. In some embodiments, a combination of arrhythmia-inducing methods can be combined.

In one aspect, provided herein is a kit comprising a multi-electrode array as described herein. In some embodiments, the kit can further comprise at least one electroconductive cell. In some embodiments, provided herein is a kit comprising a cell culture system as described herein.

A kit is any manufacture (e.g., a package or container) comprising at least one multi-electrode array according to the various embodiments herein, the manufacture being promoted, distributed, or sold as a unit for performing the methods or assays described herein. The kits described herein include reagents and/or components that permit the culture of electroconductive cells and/or the recording and/or stimulation of electrical signals associated with those cells. The kits described herein can optionally comprise additional components useful for performing the methods and assays described herein. Such reagents can include, e.g. cell culture media, growth factors, differentiation factors, buffer solutions, labels, imaging reagents, and the like. Such ingredients are known to the person skilled in the art and may vary depending on the particular cells and methods or assay to be carried out. Additionally, the kit may comprise an instruction leaflet and/or may provide information as to the relevance of the obtained results.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used herein, the term “substrate” refers to any suitable carrier material to which the cells are able to attach or adhere in order to survive and/or proliferate. In accordance with some embodiments, the cell culturing layer can be applied to the substrate to facilitate cell culturing on the cell culturing surface of the cell culturing layer.

As used herein, the term “suitable for culture”, as used in reference to a substrate for the culture of cells refers to having the necessary characteristics to allow the culture and/or maintenance of cells, e.g. for at least continuing the viability of a cell or a population of cells. A maintained population of cells will have at least a subpopulation of metabolically active cells. By way of non-limiting example, a substrate suitable for the culture of cells will not comprise any agents that are toxic to the cells; will be sterile, substantially sterile, or amenable to sterilization; and/or will be provided with a source of cell culture medium or means of containing cell culture medium in contact with the cells, etc.

The term “substantially” as used herein means for the most part, essentially the same as the character it is substantially a feature of. In some embodiments, for example, a feature which is “substantially parallel” refers to features which are at least about 60%, or preferably at least about 70% or at least about 80%, or at least about 90%, at least about 95%, at least about 97% or at least about 99% or more, or any integer between 70% and 100% similar to a parallel structure. In some embodiments, for example, a surface which is “substantially smooth” is a surface which is at least about 60%, or preferably at least about 70% or at least about 80%, or at least about 90%, at least about 95%, at least about 97% or at least about 99% or more, or any integer between 70% and 100% similar to a smooth structure.

The term “nanotextured” as used herein refers to a repeating pattern of substantially parallel grooves and ridges where the heights and depths and width of the grooves and ridges are all of sub-micron scale.

The term “anisotropic” refers to items, such as cells, being spatially organized or arranged in a direction-related manner.

As used herein, the term “grooves” refers to a relative recess in a surface having a width, length, and depth wherein the length is greater than the width. In some embodiments, a groove can be substantially linear. In some embodiments, a groove can be linear. In some embodiments, a portion of a groove can be substantially linear. In some embodiments, a portion of a groove can be linear. In some embodiments, a groove can be recessed relative to the surface of the substrate, e.g. the surface prior to introduction of grooves and/or ridges. In some embodiments, a groove can be recessed relative to a ridge.

As used herein, the term “ridges” refers to a portion of a surface which is relatively elevated and/or raised, having a width, length, and height wherein the length is greater than the width. In some embodiments, a ridge can be substantially linear. In some embodiments, a ridge can be linear. In some embodiments, a portion of a ridge can be substantially linear. In some embodiments, a portion of a ridge can be linear. In some embodiments, a ridge can be raised and/or elevated relative to the surface of the substrate, e.g. the surface prior to introduction of grooves and/or ridges. In some embodiments, a ridge can be raised and/or elevated relative to a groove.

As used herein, the term “array” refers to an order, arrangement or series of particular elements. For example, any array of grooves and ridges refers to an arrangement of multiple grooves and/or ridges while an electrode array refers to an arrangement of multiple electrodes (e.g. 2 or more electrodes, 3 or more electrodes, or 4 or more electrodes).

As used herein, the term “cell culture chamber” refers to a reservoir in or on a substrate that can retain culture medium to support the growth and/or maintenance of cells. In some embodiments, a cell culture chamber can be a well, a depression, or an area bounded by walls. In some embodiments, at least one surface of a cell culture chamber can comprise grooves and ridges. In some embodiments, a cell culture chamber can comprise a microelectrode and/or an array of microelectrodes.

As used herein, the term “biocompatible” as used herein within the context of a substrate denotes a composition that is not biologically harmful, e.g. a material that is suitable for implantation into a subject or suitable for the growth and/or maintenance of cells in vitro. A biocompatible substrate does not cause toxic or injurious effects once implanted in the subject or when in contact with cells.

The term “biodegradable” as used herein within the context of a substrate denotes a composition that is not biologically harmful and can be chemically degraded or decomposed by natural effectors (e.g., weather, soil bacteria, plants, animals).

The term “bioresorbable” as used herein within the context of a substrate refers to the ability of a material to be reabsorbed over time in the body (e.g. in vivo) so that its original presence is no longer detected once it has been reabsorbed.

The term “bioreplaceable” as used herein within the context of a substrate as used herein, and when used in the context of an implant, refers to a process where de novo growth of the endogenous tissue replaces the implant material. A bioreplacable material as disclosed herein does not provoke an immune or inflammatory response from the subject and does not induce fibrosis. A bioreplaceable material is distinguished from bioresorbable material in that bioresorbable material is not replaced by de novo growth by endogenous tissue.

As used herein, the term “microelectrode” refers to a metalized layer that can receive electrical signals from and apply electrical signals to cells and tissue through direct contact or through a dielectric material.

As used herein, the term “lead” refers to a conductive element such as wire or a film that connects an electrode to a pad or signal processing or generating circuitry.

As used herein, the term “conditioning blocks” refers to electrical components that can be used to condition the signals received from cells, such as signal amplifiers and filters.

As used herein, the term “digitally sampled” refers to measuring an amplitude of a signal at a predefined time or periodically over a period of time.

As used herein, the term “computer” refers to a general purpose computing device having one or more processors and associated memories for storing programs (e.g., sequences of instructions) and data (e.g., digital representations of signals).

As used herein, the term “stimulate” refers to contacting a cell with, or providing to a cell, a compound, signal, condition, and/or force that elicits a reaction from the cell. In some embodiments, stimulation can encompass providing an electrical impulse to a cell.

As used herein, the term “record” or “acquire” in reference to data (e.g. biopotential data) refers to storing data representative of biopotentials and/or impedances in a digital format in volatile or non-volatile memory.

As used herein, the term “interface electronics” refers to electrical components that convert electrical signals between formats or connect electronic components to enable signals to pass between them.

As used herein, the term “signal generator” refers to an electrical component that produces an electrical signal. A signal generator can be a group of components configured to produce a signal having one or more predefined characteristics (e.g., frequency and amplitude). A signal generator can include a memory device that plays back a recorded signal. A cell or tissue sample can also be used as a signal generator.

As used herein, the term “sine wave” refers to a signal that approximates a sinusoid or sine function and provides a generally periodic signal.

As used herein, the term “signal” refers to a detected electrical potential such as a biopotential or a detected impedance. The term “signal” also refers to an electrical potential that is applied to a cell or tissue to measure impedance or elicit a response.

As used herein, the term “signal recorder” refers to a device that records signals for later use or analysis.

The term “biopotentials” as used herein refers to a voltage produced by the cells cultured on the MEA substrate, particularly muscle cells or neuronal cells on the MEA substrate. Non-limiting examples of functional parameters of biopotentials can include action potential duration (ADP), wave propagation, action potential frequency, beat frequency, action potential transmission, Vmax of the action potential, contraction force, peal to peak amplitude, end diastole to peak diastole rate and the like.

As used herein, a “field potential” refers to an electrophysiological signal which is primarily the electrical current flowing through a volume of tissue. In this situation, “potential” refers to electrical potential, or voltage, in the volume of tissue. A field potential can be, e.g., a local field potential or an extracellular field potential.

As used herein, “conduction velocity” refers to the speed at which an electrochemical impulse propagates across or along a tissue, e.g. along a muscle or neuronal tissue.

As used herein, “electrophysical profile” refers to the profile (e.g. pattern, frequency, magnitude, etc) of electrical properties of a cell and/or tissue. Electrophysical profiles can include, by way of non-limiting example, voltage change profiles, electrical current profiles, action potential activity, field potentials, changes in ion concentrations or changes in biomolecules which regulate or are regulated by electrical activity in electroconductive cells.

As used herein, “reentrant wave patterns” refers to the pattern of an electrical impulse in a heart or heart tissue which travels in a small circle within the tissue instead of transiting the tissue and then stopping. These phenomenon can cause arrhythmia.

As used herein, “impedance,” when used in reference to a property of a cell and/or tissue refers to a measure of the opposition to an electrical current in that cell or tissue.

Cardiomyocytes, in the absence of electrical stimulus, will display spontaneous beating behavior, e.g. cycles of contraction. The frequency of these contractions, the spontaneous beating rate, can be measured.

As used herein “QT interval” refers to the time between the start of the Q wave and the end of the T wave in the heart's electrical cycle. As used herein, the term “electroconductive” such as in reference to a cell refers to the property of being able to conduct, generate, and/or respond to an electrical signal. Examples of electroconductive cells are neurons and muscle cells.

As used herein, the term “neuronal cell” or “neuron” refers to cells found in the nervous system that are specialized to receive, process, and transmit information as nerve signals. Neurons can include a central cell body or soma, and two types of projections: dendrites, by which, in general, the majority of neuronal signals are conveyed to the cell body; and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the nervous system.

The term “myocytes” refer to a muscle cells. Sub-categories of myocytes include, for example, skeletal myocytes, smooth muscle myocytes, cardiomyocytes, as well as ESC- and iPSC-derived myocytes.

The term “cardiomyocyte” as used herein broadly refers to a muscle cell of the heart (e.g. a cardiac muscle cell). The term cardiomyocyte includes smooth muscle cells of the heart, as well as cardiac muscle cells, which also include striated muscle cells, as well as spontaneous beating muscle cells of the heart. A cardiomyocyte will generally express on its cell surface and/or in the cytoplasm one or more cardiac-specific marker. Suitable cardiomyocyte-specific markers include, but are not limited to, cardiac troponin I, cardiac troponin-C, tropomyosin, caveolin-3, GATA-4, myosin heavy chain, myosin light chain-2a, myosin light chain-2v, ryanodine receptor, and atrial natriuretic factor.

As used herein, the term “skeletal muscle myocyte” refers to muscle cells found in skeletal muscles, e.g. voluntary muscles anchored to bone and used to effect locomotion and maintain posture.

As used herein, “smooth muscle myocyte” refers to muscle cells found within the walls of organs and structures (e.g. the esophagus, stomach, intestines, bronchi, uterus, urethra, bladder, and blood vessels) and which is not under voluntary control.

The term “myogenically committed” or “myogenic committed” refers to a cell, such as a progenitor cell, such as a myogenic progenitor cell, which differentiates into a substantially pure population of cardiac muscle cells such as cardiomyocytes.

The terms “cardiac progenitor cell” and “CPC” are used interchangeably herein to refer to a progenitor cell which is capable of proliferation and giving rise to more progenitor cells having the ability to generate a large number of mother cells that can in turn give rise to differentiated, or differentiable daughter cells which can eventually terminally differentiate primarily into cells of the heart tissue, including endothelial lineages and muscle lineages (smooth, cardiac and skeletal muscles).

The term “progenitor cell” is used herein to refers to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. As indicated above, stem cells have been found resident in virtually every tissue. Accordingly, the technology described herein appreciates that stem cell populations can be isolated from virtually any animal tissue.

As used herein, the term “adult cell” refers to a cell found throughout the body after embryonic development.

As used herein, the terms “iPS cell” and “induced pluripotent stem cell” are used interchangeably and refers to a pluripotent cell artificially derived (e.g., induced by complete or partial reversal) from a differentiated somatic cell (i.e. from a non-pluripotent cell). A pluripotent cell can differentiate to cells of all three developmental germ layers.

The term “derived from” used in the context of a cell derived from another cell means that a cell has stemmed from (e.g. changed from or was produced by) a cell which is a different cell type. In some instances, for example, a cell derived from an iPS cell refers to a cell which has differentiated from an iPS cell. Alternatively, a cell can be converted from one cell type to a different cell type by a process referred to as transdifferention or direct reprogramming. Alternatively, in the terms of iPS cells, a cell (e.g. an iPS cell) can be derived from a differentiated cell by a process referred to in the art as dedifferentiation or reprogramming.

The term “pluripotent” as used herein refers to a cell that can give rise to any type of cell in the body except germ line cells. The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages. Pluripotency is also evidenced by the expression of embryonic stem (ES) cell markers, although the preferred test for pluripotency is the demonstration of the capacity to differentiate into cells of all three germ layers, as detected using, for example, a nude mouse teratoma formation assay. iPS cells are pluripotent cells. Pluripotent cells undergo further differentiation into multipotent cells that are committed to give rise to cells that have a particular function. For example, multipotent cardiovascular stem cells give rise to the cells of the heart, including cardiomyocytes, as well as other cells involved in the vasculature of the heart. Cell useful for in vitro differentiation to myocytes or cardiomyocytes as disclosed herein include, for example, iPS cells as well as multipotent cardiovascular stem cells. A major benefit of the use of iPSC or other stem cells to generate myocytes or cardiomyocytes for the compositions and methods as disclosed herein is the ability to prepare large numbers of such cells and propagate them, e.g., from a specific human patient or subject. This is in contrast to methods, compositions that rely upon the isolation and use of adult cardiac cells.

The term “phenotype” refers to one or a number of total biological characteristics that define the cell or organism under a particular set of environmental conditions and factors, regardless of the actual genotype.

As used herein, “bioactive agents” or “bioactive materials” refer to naturally occurring biological materials, for example, extracellular matrix materials such as fibronectin, vitronection, and laminin; cytokins; and growth factors and differentiation factors that have a biological effect on a biological cell, tissue or organ. “Bioactive agents” or “bioactive materials” also refer to artificially synthesized materials, molecules or compounds that have a biological effect on a biological cell, tissue or organ. The molecular weights of the bioactive agent can vary from very low (e.g. small molecules, 200-500 Daltons) to very high (e.g. plasmid DNA, 2,000,000 Daltons). In some embodiments, the bioactive agent is a small molecule. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD). In some embodiments, a small molecule can have a molecular weight of less than 3 kD. In some embodiments, a small molecule can have a molecular weight of less than 2 kD. In some embodiments, a small molecule can have a molecular weight of less than 1 kD. In some embodiments, a small molecule can have a molecular weight of less than 700 D.

The term “isolated” or “enriching” or “partially purified” as used herein refers, in the case of an in vitro-differentiated cardiomyocyte is separated from at least one other cell type. The term “enriching” is used synonymously with “isolating” cells, and means that the yield (fraction) of cells of one type is increased by at least 10% over the fraction of cells of that type in the starting culture or preparation.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a preparation of one or more partially and/or terminally differentiated cell types, such as immature cardiomyocytes, refer to a population of cells that contain fewer than about 20%, more preferably fewer than about 15%, 10%, 8%, 7%, most preferably fewer than about 5%, 4%, 3%, 2%, 1%, or less than 1%, of cells that are immature cardiomyocytes or immature cardiomyocytes progeny.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “lineages” as used herein refers to a term to describe cells with a common ancestry, for example cells that are derived from the same cardiovascular stem cell or other stem cell, or cells with a common developmental fate. By way of an example only, when referring to a cell that is of endoderm origin or is “endodermal linage,” this means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The term “contacting” or “contact” as used herein in connection with contacting a mature cardiomyocyte, either present on a substrate, or in the absence of a support, with an agent as described herein, includes subjecting the cell to a culture medium which comprises that agent. The term “modulate” is used consistently with its use in the art, e.g., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “reprogramming” as used herein refers to the transition of a differentiated cell to become a pluripotent or multipotent progenitor cell. Stated another way, the term reprogramming refers to the transition of a differentiated cell to an earlier developmental phenotype or developmental stage. A “reprogrammed cell” is a cell that has reversed or retraced all, or part of its developmental differentiation pathway to become a progenitor cell. Thus, a differentiated cell (which can only produce daughter cells of a predetermined phenotype or cell linage) or a terminally differentiated cell can be reprogrammed to an earlier developmental stage and become a progenitor cell, which can both self renew and give rise to differentiated or undifferentiated daughter cells. The daughter cells themselves can be induced to proliferate and produce progeny that subsequently differentiate into one or more mature cell types, while also retaining one or more cells with parental developmental potential. The term reprogramming is also commonly referred to as retrodifferentiation or dedifferentiation in the art. A “reprogrammed cell” is also sometimes referred to in the art as an “induced pluripotent stem” (iPS) cell.

In the context of cell ontogeny, the term “differentiated”, or “differentiating” is a relative term. A “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an atrial precursor), and then to an end-stage differentiated cell, such as atrial cardiomyocytes or smooth muscle cells, which play a characteristic role in a certain tissue type, and may or may not retain the capacity to proliferate further. The term “differentiated cell” refers to any primary cell that is not, in its native form, pluripotent as that term is defined herein. The term a “differentiated cell” also encompasses cells that are partially differentiated, such as multipotent cells, or cells that are stable non-pluripotent partially reprogrammed cells. In some embodiments, a differentiated cell is a cell that is a stable intermediate cell, such as a non-pluripotent partially reprogrammed cell. It should be noted that placing many primary cells in culture can lead to some loss of fully differentiated characteristics. However, simply culturing such primary cells, e.g., after removal or isolateion from a tissue or organism does not render these cells non-differentiated cells (e.g. undifferentiated cells) or pluripotent cells. The transition of a differentiated cell (including stable non-pluripotent partially reprogrammed cell intermediates) to pluripotency requires a reprogramming stimulus beyond the stimuli that lead to partial loss of differentiated character in culture.

The term “differentiation” as referred to herein refers to the process whereby a cell moves further down the developmental pathway and begins expressing markers and phenotypic characteristics known to be associated with a cell that are more specialized and closer to becoming terminally differentiated cells. The pathway along which cells progress from a less committed cell to a cell that is increasingly committed to a particular cell type, and eventually to a terminally differentiated cell is referred to as progressive differentiation or progressive commitment. Cell which are more specialized (e.g., have begun to progress along a path of progressive differentiation) but not yet terminally differentiated are referred to as partially differentiated. Differentiation is a developmental process whereby cells assume a more specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, in the context of this specification, the terms “differentiation” or “differentiated” refer to cells that are more specialized in their fate or function than at one time in their development. For example in the context of this application, a differentiated cell includes a ventricular cardiomyocyte which has differentiated from cardiovascular progenitor cell, where such cardiovascular progenitor cell can in some instances be derived from the differentiation of an ES cell, or alternatively from the differentiation of an induced pluripotent stem (iPS) cell, or in some embodiments from a human ES cell line. Thus, while such a ventricular cardiomyocyte cell is more specialized than the time in which it had the phenotype of a cardiovascular progenitor cell, it can also be less specialized as compared to when the cell existed as a mature cell from which the iPS cell was derived (e.g. prior to the reprogramming of the cell to form the iPS cell).

A cell that is “differentiated” relative to a progenitor cell has one or more phenotypic differences relative to that progenitor cell and characteristic of a more mature or specialized cell type. Phenotypic differences include, but are not limited to morphologic differences and differences in gene expression and biological activity, including not only the presence or absence of an expressed marker, but also differences in the amount of a marker and differences in the co-expression patterns of a set of markers.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “tissue” refers to a group or layer of similarly specialized cells which together perform certain special functions. The term “tissue-specific” refers to a source or defining characteristic of cells from a specific tissue.

The term “genetically modified” as used herein refers to a cell or organism in which genetic information or material has been modified by human manipulation. Modification can be effectuated by chemical, physical, viral or stress-induced or other means, including introduction exogenous nucleic acid through any standard means, such as transfection, such that the cell or organism has acquired a new characteristic, phenotype, genotype, and/or gene expression product, including but not limited to a gene marker, a gene product, and/or an mRNA, to endow the original cell or organism, at a genetic level, with a function, characteristic, or genetic element not present in non-genetically modified, non-selected counterpart cells or entities.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A multi-electrode array comprising:

a cell culture chamber, the cell culture chamber including a cell culture layer, the cell culture layer including a cell culturing surface and being adapted to support culturing of cells thereon;

the cell culture surface including a plurality of nanogrooves;

the cell culture layer including a plurality of electrodes, each electrode including a lead for electrically connecting the electrode to an external electrical circuit.
2. The multi-electrode array according to paragraph 1 wherein each of the plurality of nanogrooves extends substantially parallel to a first axis.
3. The multi-electrode array according to paragraph 1 wherein at least one of the plurality of nanogrooves is substantially curved.
4. The multi-electrode array according to paragraph 1 wherein at least one of the plurality of nanogrooves includes a portion that has an irregular width.
5. The multi-electrode array according to paragraph 1 wherein at least one of the plurality of nanogrooves includes a portion that has an irregular depth.
6. The multi-electrode array according to paragraph 1 wherein at least one of the plurality of nanogrooves includes a portion that is discontinuous.
7. The multi-electrode array according to paragraph 1 wherein at least one of the plurality of nanogrooves branches to form two nanogrooves.
8. The multi-electrode array according to paragraph 1 wherein at least one of the plurality of nanogrooves includes a first portion that extends straight along a first axis, a second portion that is substantially curved.
9. The multi-electrode array according to paragraph 8 wherein the at least one of the plurality of nanogrooves further includes a third portion that extends straight along the first axis.
10. The multi-electrode array according to paragraph 1 wherein at least one of the nanogrooves includes a substantially uniform width.
11. The multi-electrode array according to paragraph 1 wherein at least one of the nanogrooves is between 100 nm and 1000 nm wide.
12. The multi-electrode array according to paragraph 1 wherein at least one of the nanogrooves includes a substantially uniform depth.
13. The multi-electrode array according to paragraph 1 wherein at least one of the nanogrooves is between 100 nm and 1000 nm deep.
14. The multi-electrode array according to paragraph 1 wherein at least a portion of one electrode is affixed to a portion of the cell culture surface of the cell culture layer.
15. The multi-electrode array according to paragraph 14 the one electrode further includes a via that extends through the cell culture layer that electrically connects the one electrode to a lead.
16. The multi-electrode array according to paragraph 1 wherein at least a portion of one electrode follows a surface contour of at least one of the plurality of the nanogrooves.
17. The multi-electrode array according to paragraph 16 the one electrode further includes a via that extends through the cell culture layer that electrically connects the one electrode to a lead.
18. The multi-electrode array according to paragraph 1 wherein at least a portion of one electrode extends below the cell culture surface of the cell culture layer.
19. The multi-electrode array according to paragraph 18 wherein the at least a portion of one electrode extends between the substrate and the cell culture layer.
20. The multi-electrode array according to paragraph 1 wherein at least one of the nanogrooves includes a bottom surface and at least a portion of one electrode forms at least a portion of the bottom surface of the at least one nanogroove.
21. The multi-electrode array according to paragraph 1 wherein the external electrical circuit includes at least one of an amplifier, a recording device and an analysis device.
22. The multi-electrode array according to any of paragraphs 1-21, comprising a plurality of cell culture chambers.
23. A method of making a multi-electrode array comprising:

providing a cell culture chamber defining a first surface having a plurality of electrodes spaced apart over the first surface;

forming a cell culture layer on the first surface, the cell culture layer defining a [second] cell culture surface; and
forming at least one nanogroove in the cell culture surface.
24. The method according to paragraph 23 comprising forming a plurality of nanogrooves in the cell culture surface.
25. The method according to paragraph 23 wherein the at least one nanogroove is formed by capillary force lithography.
26. The method according to paragraph 23 wherein the at least one nanogroove is formed by masking the cell culture layer and etching the at least one nanogroove.
27. The method according to paragraph 26 wherein etching includes one or more of laser etching, chemical etching, dry etching and reactive ion etching.
28. The method according to paragraph 23 wherein the at least one nanogroove is formed by stamping.
29. The method according to paragraph 23 wherein the cell culture layer is between 2 μm and 15 μm thick.
30. The method according to paragraph 23 wherein the cell culture layer is formed by laminating a polymer layer on to the first surface of the cell culture chamber.
31. The method according to paragraph 23 wherein the cell culture layer is formed by casting a polymer layer on to the first surface of the cell culture chamber.
32. The method according to paragraph 23 wherein the cell culture layer includes at least one of Polydimethylsiloxane (PDMS), Polyurethane (PU), polyethylene glycol (PEG), and other hydrogels that are UV curable.
33. The method according to paragraph 23 wherein the cell culture layer includes a flexible biocompatible material.
34. A method of making a multi-electrode array comprising:

forming at least one conductive lead on a first surface of a base material;

forming a cell culture layer on the first surface of the base material, the cell culture layer forming a cell culture surface, the cell culture surface including a plurality of nanogrooves;

applying a first mask to the cell culture surface and etching at least one hole for a via in the cell culture material, the at least one hole aligning with the at least one conductive lead;

applying a conductive material in the at least one hole to form a via;

applying a second mask to the cell culture surface and forming at least one electrode on the cell culture surface, the at least one electrode making electrical contact with the conductive lead through the via.
34. The method according to paragraph 33 further comprising forming chamber walls on the cell culture surface to form a cell culture chamber around the at least one electrode.
35. The method according to paragraph 33 wherein the base material is glass.
36. The method according to paragraph 33 wherein the cell culture layer includes a flexible biocompatible material.
37. The method according to paragraph 33 wherein the cell culture layer includes polydimethylsiloxane (PDMS), polyurethane (PUA), poly ethylene glycol (PEG), and other hydrogels that are UV curable.
38. The method according to paragraph 33 wherein the cell culture layer is between 2 μm and 15 μm thick.
39. The method according to paragraph 33 wherein the cell culture layer is coated with either or a combination of gelatin, fibronectin, laminin, or engineered peptides.
40. The method according to paragraph 33 wherein the nanogrooves are formed by capillary force lithography-based nano-patterning.
41. The method according to paragraph 33 wherein the nanogrooves are formed by stamping.
42. The method according to paragraph 33 wherein the nanogrooves are formed by etching.
43. The method according to paragraph 33 wherein at least one of the nanogrooves is between 100 nm and 1000 nm wide.
44. The method according to paragraph 33 wherein at least one of the nanogrooves is between 100 nm and 1000 nm deep.
45. The method according to paragraph 33 wherein the at least one electrode is formed by sputtering a conductive material on the cell culture surface.
46. The method according to paragraph 45 wherein the conductive material includes at least one of chromium, gold, titanium, oxides and nitrides of above materials, stainless steel, tungsten, or copper.
47. The method according to paragraph 45 wherein the conductive material includes modified structures that include at least one of carbon nanotubes, gold pillars, and nanocavities.
48. A cell culture system comprising a multi-electrode array of any of paragraphs 1-22 further comprising an electroconductive cell present on the cell culture surface.
49. The system of paragraph 48, wherein the electroconductive cells are muscle cells.
50. The system of paragraph 49, wherein the muscle cells are selected from the group consisting of:

cardiomyocytes; skeletal muscle myocytes; and smooth muscle myocytes.
51. The system of paragraph 48, wherein the electroconductive cells are neuronal cells.
52. The system of any of paragraphs 48-51, comprising a plurality of types of electroconductive cells.
53. An assay for measuring the effect of an agent on electroconductive cells, the assay comprising:

contacting a system of any of paragraphs 48-52 with the agent;

and measuring the growth, viability, or activity of the electroconductive cells.
54. The assay of paragraph 53, wherein the measured activity of the electroconductive cells is selected from the group consisting of:
   field potential; field potential profiles; field potential duration; QT interval length, conduction velocity; electrophysical profile; spontaneous beating rate; reentrant wave patterns; and impedance.
55. The assay of any of paragraphs 53-54, wherein the electroconductive cells comprise cells obtained from or descended from cells obtained from a subject with a cardiac disease.
56. The assay of any of paragraphs 53-55, wherein the electroconductive cells have been modified to comprise a mutation associated with a cardiac disease.
57. The assay of any of paragraphs 53-56, wherein the assay further comprises inducing arrhythmia in the cells and the measuring the ability of the agent to restore normal field potential properties.
58. The assay of paragraph 57, wherein arrhythmia can be reduced by a method selected from the group consisting of:

culturing the cells on a multi-electrode array comprising irregular nanogrooves;

contacting the cells with an arrhythmia-inducing agent; and

providing irregular electrical stimulation via the multi-electrode array.
59. A kit comprising the multi-electrode array of any of paragraphs 1-22.
60. A kit of paragraph 59, further comprising at least one electroconductive cell.

### EXAMPLES

### Example 1

Described herein is the design and implementation of a Multi-Electrode Array (MEA) integrated with a nanotopographically patterned substrate. The MEA described herein can accommodate an in-vivo environment, including local micro- and nano-scale molecular and topographic patterns provided by, for instance, complex and well-defined structures of the extracellular matrix (ECM). Unlike existing MEAs, the proposed MEA acquires biological signals from cells that sit on nano-scale grooves, which mimic the structural and mechanical cues present in the in-vivo ECM environment. The MEA described herein permits the acquisition of biological signals of cells that closely matches with that in in-vivo environments.

Specifically contemplated herein is the design, implementation, and evaluation of a single-well MEA on nano-grooves; the development of a multi-well MEA on nano-grooves; and the implementation of the interface of biopotentials and impedance measurements

In-vitro acquisition of electrophysiological signals of cells provides an effective method to understand cell growth and behavior. Existing devices have an array of electrodes, either planar or 3d, on a flat rigid surface and directly contact cells' outer membrane to collect electrophysiological signals. However, it is very challenging to equate electrophysiological signals acquired in-vitro to those in in-vivo environments, using the existing devices. This challenge is primarily because cells on the existing devises experience far different environments in-vivo. Recent studies on the cell sensitivity to the nano-scale features of the cell/tissue environment elucidates how cells respond to nano-scale mechano-features, often encountered in the in-vivo environment. Thus, described herein is the combination of the nano-scale features and MEA to accomplish “MEA in in-vivo mimicking environments”.

The devices described herein can comprise a soft substrate, such as PDMS Polydimethylsiloxane (PDMS) or Polyurethane (PU), having 100s of nm features, commonly known as “nanogrooves”, to mimic in-vivo-like environments. The MEA electrodes sit directly on the nanogrooves, covering the contour of delicate nano-scale features, and collect electrophysiological signals from cells. Described herein is the development of i) a single-well MEA, having up to 128 channels, ii) a multi-well MEA, having up to 96 wells, on a nanogrooved substrate and iii) an electronic interface to acquire biopotentials and impedance of cells. The devices described herein permit the acquisition of electrophysiological data of cells in in-vivo mimicking environments.

Described herein is the development and evaluation of integrated MEAs on a nano-scale soft substrate. This approach is unique and useful as the integrated MEA offers biosignals of cells in in-vivo-like environments, which cannot be obtained using existing MEAs. In its ultimate form, the 96-well-128-channel MEA system delivers high throughput biosignals of cells in a compact platform. The results from this research can offer significantly enhanced biosignals from cells, largely different from prior methods, which accurately describe behavior of cells in in-vivo microenvironments.

Existing Multi-Electrode-Array (MEA) and Limitations

TABLE 1
Existing MEAs and their specifications

| | | Spacing/ | Electrode/ | Noise | |
|---|---|---|---|---|---|
| | # of | Diameter | Insulation | Level | |
| | Electrodes | [μm] | Material | [μV] | Note |
| Multichannel | 32-256 | 30-1000/ | TiN, ITO, Au/ | <0.8 | Perfusion, temperature control, |
| Systems [14] | | 10-100 | SiN | | enhanced optical imaging, integrated |
| | | | | | stimulation, up to 9 wells |
| Qwane | 60-256 | 100-1000/ | Au, Pt, ITO/ | 10-35 | 3D electrodes (25-80 μm height), |
| Biosciences | | 30-80 | SU-8 | | integrated stimulation, porous |
| [15] | | | | | substrates, up to 9 wells |
| MED64 [16] | 64 | 100-450/ | ITO or Pt/ | 1 | Integrated stimulation, up to 4 wells |
| | | 20 or 50 | Acrylate resin | | |
| Axion | 64 | 200/30 | Nanoporous | N/A | Up to 96 well systems with plate |
| Biosystems | | | Au/SU-8 or | | recognition (high throughput) |
| [17] | | | SiO₂ | | |

The first fabricated MEAs for use in in-vitro cell cultures were reported in the early 1970s and were used to acquire field potentials from cardiac cells [11]. From the 1980s onwards, several significant advancements in MEA technology were made and included: integration of transistors as electrically-sensitive electrodes and active circuitry onboard the MEA for increasing signal to noise ratio, higher density of electrodes to increase the spatial resolution of recordings, integration of MEAs with dye-sensitive or optical monitoring techniques, conformal substrates, long term viability, etc. Many MEAs were commercialized in the early 1990s and 2000s, including Multichannel Systems (Germany), Ayanda (Switzerland, now Qwane Biosciences), and Panasonic (USA, now Alpha Med Scientific's MED64). Table 1 summarizes the specifications of several MEAs. MEA recording electrodes may have a density of up to 256, diameters in the range of 10-100s of μm, inter-electrode spacing between 30-1000 μm, and have different geometrical configurations (hexagonal, rectangular, or other types of array geometries) [12, 13].

It is contemplated herein that higher densities, smaller spacing and higher number of electrodes, is helpful in application for cardiac cell cultures as they can resolve subtle changes in local field potentials. This not only provides higher spatial accuracy, but can also reveal considerably more information that could not be extracted with lower spatial density such as other physiological effects or underlying changes in the cells [12]. To further increase the statistical accuracy of the recorded data, higher throughput can be required, and such features can be obtained by incorporating multiple wells, such that multiple cell culture populations may be recorded simultaneously.

Cell Sensitivity to the Nano-Scale Features of the Cell/Tissue Environment.

The Extracellular Matrix (ECM) can play a plethora of roles in control of cell and tissue function. For instance, it provides cells with a suitable adhesion substratum and can supply cells with a variety of embedded chemical cues. A somewhat less appreciated role of ECM is its ability to impose mechanical cues, such as the degree of the matrix rigidity or the size, orientation, deformability and local density of the ECM fibers [2]. As cells are commonly analyzed while incubated on flat and infinitely rigid surfaces characteristic of Petri dishes or flasks, the putative effects of softer, more dynamic and flexible mechanical environment, characteristic of the ECM in a variety of tissues, are frequently ignored. However, rapidly accumulating evidence strongly suggests that most if not all mammalian cell types are exquisitely sensitive to the mechanical features of the adhesion substratum, and can modify a variety of phenotypic features as a function of changes in the mechanical milieu [18]. In particular, utilization of engineered polymer gels suggests that the rigidity and nano-structure of the surrounding or underlying matrices can affect such fundamental cell properties and functions as the cell shape and orientation, the rates and directionality of cell movement and division as well as the cell fate [19].

The inventors have found that, in particular, multiple cell types can sense the variation of the density of micro- and nano-scale substratum features (e.g., nano-ridges and nano-pillars), thus displaying the topography sensing or ‘topotaxis’ [2-4]. Additionally, monolayers of rat cardiomyocytes can display enhanced force generation and action potential propagation as a function of the nano-topography feature density on the scale varying from 100 to 800 nm, suggesting extremely sensitive nano-sensory capacity in these cardiac cell and tissue models (FIGS. 12A-12C) [3].

It is contemplated herein that this sensitivity indicates that the naturally occurring nanoscale ECM can be a powerful guidance cue regulating not only cell alignment into anisotropic arrays, but also the fine details of the tissue structure and function. Unfortunately, the extent and importance of nanotopography in directing the cells to establish the physiological structure and function of the tissue for its potential clinical relevance remain poorly understood.

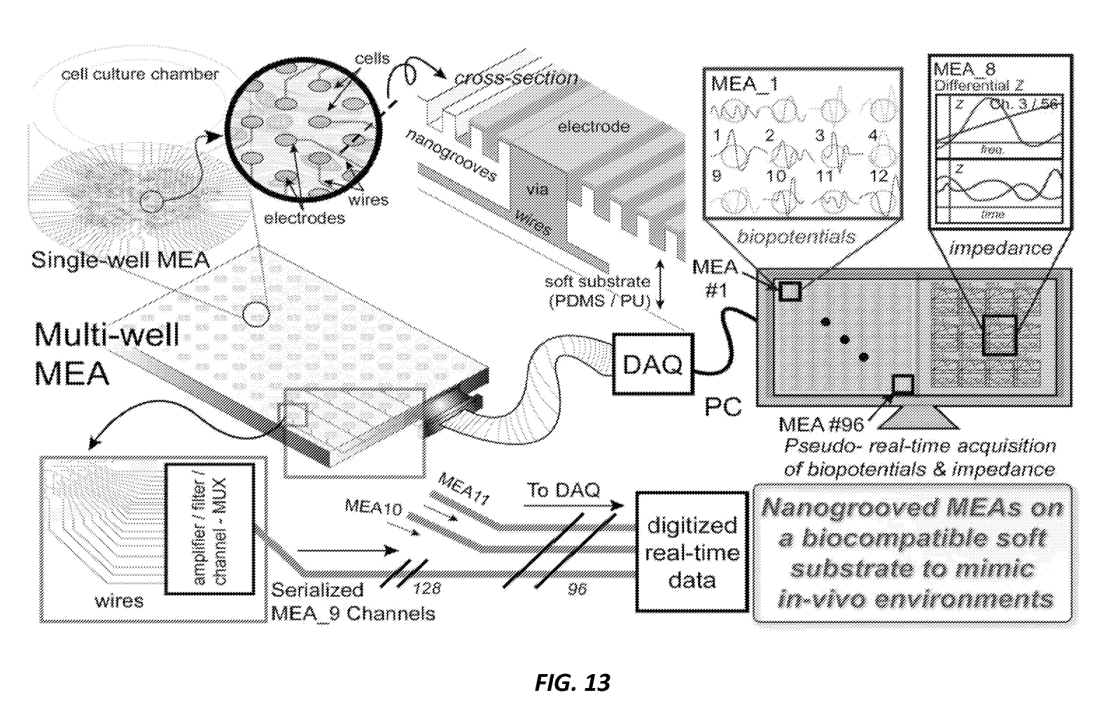

Described herein is the development of a nano-textured MEA device and characterization of electrophysiological properties of cardiac cells using the MEA. The integrated MEA permits the acquisition of electrophysiological data of cells in in-vivo mimicking environments that existing MEAs cannot provide. FIG. 13 illustrates the overall schematic of an exemplary embodiment of the in-vivo mimicking MEA based system described herein. The MEA can be integrated with a flexible biocompatible layer (e.g., Polydimethylsiloxane or Polyurethane), that includes nano-meter scale grooves. The nanogrooves can be 100 nm to 1000 nm in width, pitch, and/or depth, providing mechanical cues to the cells that are cultured on top of the grooves. The nanogrooves can be formed by capillary force lithography-based nanopatterning or other fabrication techniques such as etching or 3D printing to create micro-/nano-patterning. The PDMS or PU layer can be mounted on a glass slide or other base substrate. The MEA can be directly integrated with the nanogrooves. Electrical wires or leads can be embedded between the PDMS or PU layer and glass slide. The electrodes can be formed on the nanogrooves and the vias can be formed through the PDMS or PU layer to connect the electrodes to the wires. The electrodes can follow the contour of nanogrooves, which allow seamless interface between cells and the nano-scale in-vivo mimicking environment. The use of a glass slide or other transparent or translucent base substrate material allows fluorescent visualization and helps lower parasitics associated with electrical wires.

As shown in FIG. 13, the MEA device can be included in a system that captures biopotential signals and impedances from individual electrodes, stores the signals in a computer for future analysis. The MEA device according to the invention can include one or more wells or chambers, each including an array of electrodes on or adjacent to a cell culturing surface. Each of the electrodes can include a lead or wire to connect the electrode either directly or indirectly to external circuitry. One or more of the leads or wires can be connected to one or more signal conditioning devices, such as, amplifiers, filters, and signal processors. Two or more of the leads or wires can be connected to a channel multiplexer that enable one or more of the signals to be time division multiplexed or frequency division multiplexed. The signals received from the electrodes can be digitized using an analog to digital converter to convert the signal (e.g., biopotential and impedance) amplitude to a digital value that can stored and presented visually on a computer. The signals received from two or more electrodes can be stored and presented, synchronized in time.

The following sections describe additional details of the MEA integrated with nanogrooves. Presented herein are results on cells growth on nanogrooves and MEA attachment on the nanogrooves. Design, implementation, and evaluation of a single-well MEA on nano-grooves are also described in herein.

The single-well MEA described herein permits the acquisition of electrophysiological signal from cells on an in-vivo mimicking environment. The single-well MEA can be extended to multi-well MEA to characterize cells in various in-vivo like environments and to increase the throughput. Specifically contemplated herein is the development of up to a 96-well MEA system. Finally, the electrical interface of the proposed MEA is discussed herein. The interface is based upon a standard data acquisition instrument/software (LabVIEW) and is customizable to specific needs of users.

Results

This section describes results of i) nano-grooves on a flexible substrate and ii) MEA formation on the nano-grooves. Nano-grooves having 100s of nm features were formed on a PDMS or PU substrate and physiological responses from cardiac cells were observed. According to the observation, the nano-grooved surface significantly promotes cardiomyocyte maturation and aligns the myocytes into a functional monolayer. Small-sized array (up to 3×5) of MEA was formed on top of the nano-grooves to demonstrate technical feasibility of forming MEA on the delicate features. Impedance measurements showed the MEA should be compatible to biological signal acquisition.

Nano-Grooves on a Flexible Substrate.

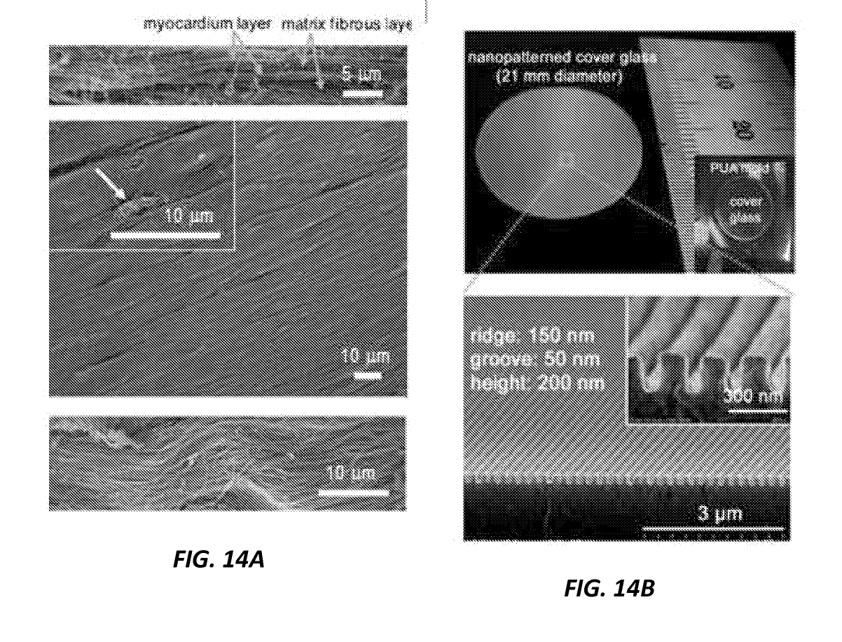

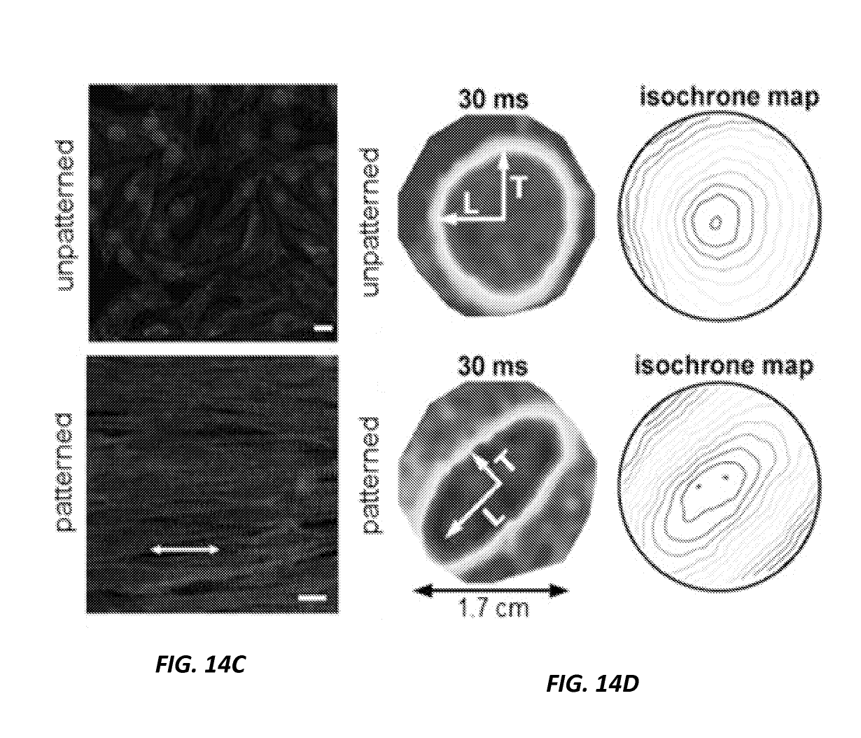

Described herein is a facile and robust method to produce a nanotopographically-controlled model of myocardium mimicking in-vivo ventricular organization [3]. Heart tissue has complex structural organization on multiple length scales, from macro- to nano-. On the macro-scale of cell sheets constituting the myocardium, myocytes and myofibrils are aligned congruently, providing a natural direction for exertion of contractile forces and a defined axis for the propagation of action potentials. In an ultrastructural analysis of the native rat myocardium, the arrangement of mutually aligned cardiomyocytes was found to correlate strongly with the direction of the fibers of the underlying ECM (FIG. 14A). This indicates a role for ECM as a natural nano-scale cue for cardiac tissue organization and development. Force lithography-based nanofabrication (CFL) techniques have been developed [2, 3, 20-23] to recapitulate the native matrix nanotopography (FIG. 14B). Cardiomyocytes lose their native organization and adopt random distribution when cultured in vitro using common techniques (FIG. 14C), potentially compromising many of their physiological properties. In contrast, cells on patterned surfaces assume markedly aligned morphology parallel to the direction of the underlying nanogrooves (FIG. 14C). Interestingly, optical mapping analysis also demonsrated that action potential propagation occurs in an anisotropic manner, and with an increased velocity in the direction of the aligned cells (FIG. 14D).

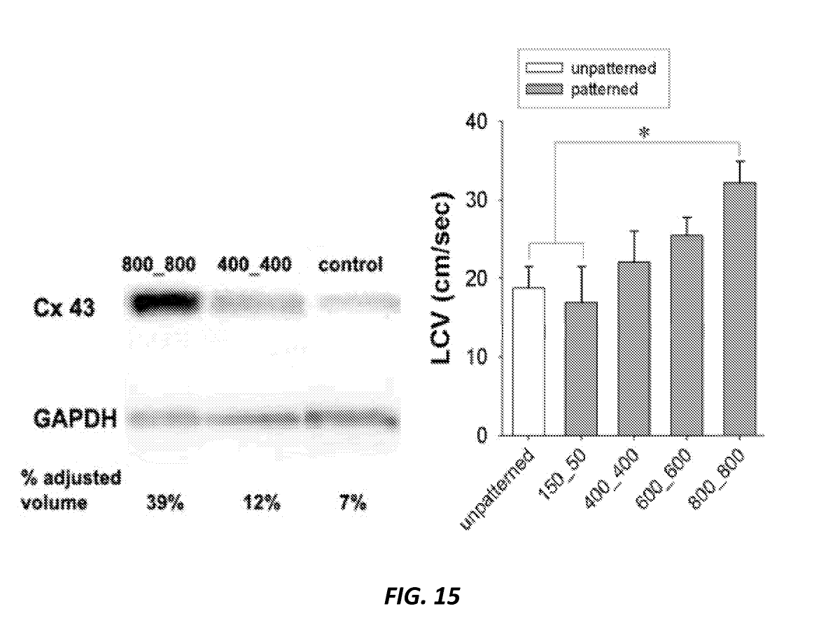

In addition, the cardiomyocytes are highly sensitive to the exact nanofeature sizes presented by the underlying matrix modulating their cell area and length. Importantly, the expression levels of gap junction protein connexin-43 were also dependent on the size of grooves and ridge widths (FIG. 15 (left)). This correlated with a higher observed conduction velocity, as feature sizes were increased to 800_{—}800 nm (groove width_ridge width) (right). Together, these results demonstrate anisotropic nanotopography can guide self-assembly of cardiomyocyte monolayers into an aligned architecture that mimics the structure of native myocardial tissue. Moreover, these results indicate that the specifics of the nanotopographic pattern can control the average cell size, cell-cell coupling, and electrophysiological properties [3], thereby potentially controlling hypertrophy and maturation.

MEA Integration with Nano-Grooves.

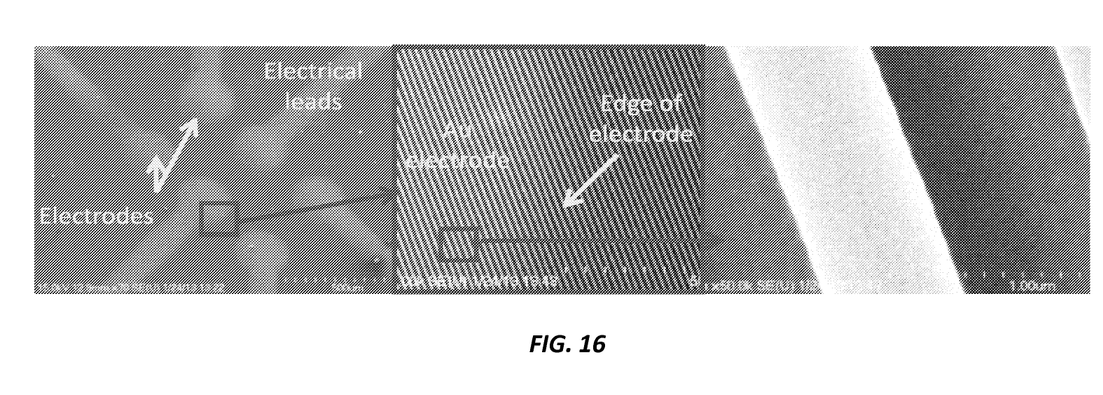

In order to explore a potential possibility of integrating MEA on nanogrooves, the MEA was directly formed on top of nanogrooves (FIG. 16). In accordance with some embodiments, the nanogrooves are formed in a PDMS layer and the thermal budget (e.g., the ability to apply heat) in subsequent steps is largely limited. In accordance with some embodiments, a shadow mask can be used to cover the MEA cell culturing layer and a thin film of Cr/Au (e.g., 40-70 nm) can be sputtered through the shadow mask on the nanogrooves. The size of the designed arrays range from 2×2 to 3×6, and FIG. 16 (left) shows a visual image of a 2×3 array. The edge of Cr/Au electrode is clearly visible FIG. 16 (middle) and all the sharp periodic feature of nano-grooves is well maintained after the deposition FIG. 16 (right).

Measurements of Exemplary Embodiment of MEA.

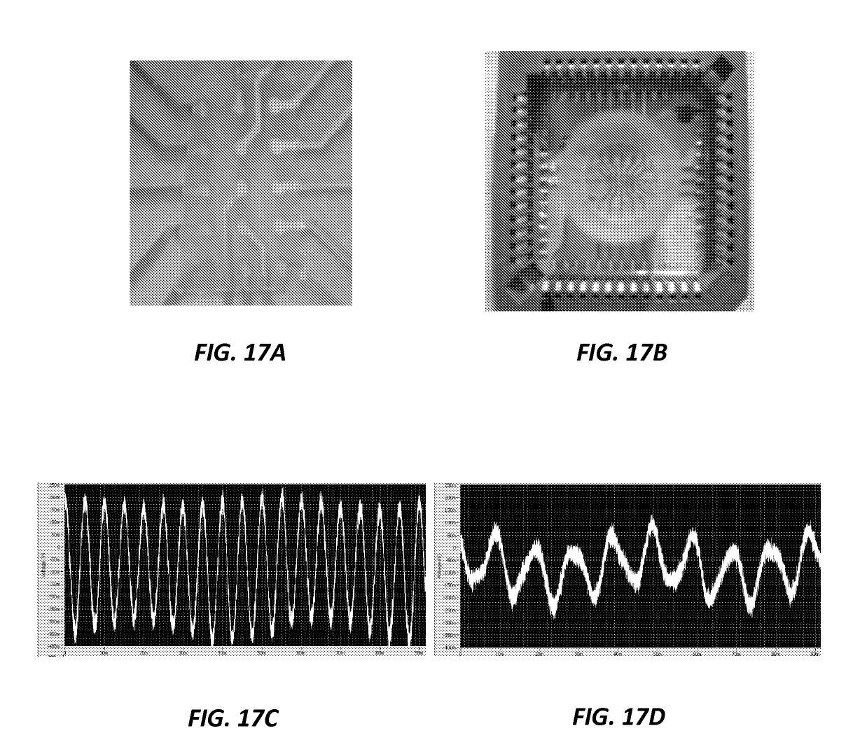

As electrodes/leads are conformal to the nanogroove profile, the resistance is a function of orientation of nanogrooves. The measured resistances of leads aligned parallel and perpendicular to the nanogrooves are 10-13Ω and 20-27Ω, respectively, matching well with the geometrical calculation. In order to provide electrical isolation among MEA electrodes, a thin (˜300 nm) biocompatible insulator, parylene-C, was deposited on top of the electrical leads, leaving the electrodes uncovered. The deposition process was performed at room temperature to preserve the delicate features of the nanogrooves. The parylene-C layer covers red-colored leads and green-colored electrodes are uncovered to allow for exposure to cells (FIG. 17A). After the deposition, a culture chamber was attached on the chip using a UV-curable biocompatible adhesive (PU 78) (FIG. 17B). FIG. 17C shows temporal signal from the MEA using emulated input from a signal generator (87.7 μV at 200 Hz), acquired from a pin located at the bottom of the wiring socket. The temporal signal was amplified and filtered to remove unwanted noise. Yet, 60 Hz noise still exists, which needs to be removed by shielding or providing a well-grounded source. The 60 Hz noise becomes clearer as the input amplitude decreases to 29.25 μV at 100 Hz (FIG. 17D). Frequency response as a function of orientation of nanogrooves was performed as well and no signal variation was observed as a function of the orientation. This is an expected result as the minute difference in resistance and capacitance associated with the electrodes/leads unlikely influence the biosignal acquisition.

Lessons from the Exemplary Embodiment Single Well MEA.

The results described in the foregoing paragraph are promising to explore the proposed MEA integrated with nanogrooves. Specifically contemplated herein are additional embodiments. The isolation layer electrically isolates electrodes of MEA. Parylene-C film primarily because parylene-C is a proven biocompatible material and is deposited in a room temperature chemical vapor deposition process, to facilitate the cell growth and to maintain fine features of nanogrooves, respectively. It is very difficult to finely pattern the film without compromising the nano-grooves. As the size of the array increases (up to a 3×6 array was tested) the isolation among electrodes becomes more critical. It is specifically contemplated herein that in some embodiments, electrical wires can be embedded underneath the nano-grooves and the electrodes left on top of them using through-vias.

It is specifically contemplated herein that in some embodiments, the devices described herein can be high-throughput or high-throughput-compatible. A multi-well MEA can be advantageous for certain applications, including drug assessment. The number of wells can be fairly large, for example, up to 96 wells. The exemplary embodiment of MEA described above herein, e.g. in FIGS. 17A-17B) contains only one culture chamber on a 2×2 to 3×6 array, which significantly limits the use of MEA for high-throughput applications. In some embodiments, an array of wells can be formed directly on top of the array of MEAs using hybrid integration of an array of wells with the nano-manufactured array of MEAs.

When the array becomes large, for example, up to 128 channels per well and 96 wells, the total number of channels becomes enormous, that is, 128×96=12,288. It will be challenging to collect signals from all of these channels and retrieve “real-time” like signals. It is contemplated herein that a systematic interface can be employed, allowing pseudo real-time acquisition of biosignals.

Single-Well MEA on Nano-Grooves

Design and Implementation of a Single-Well MEA.

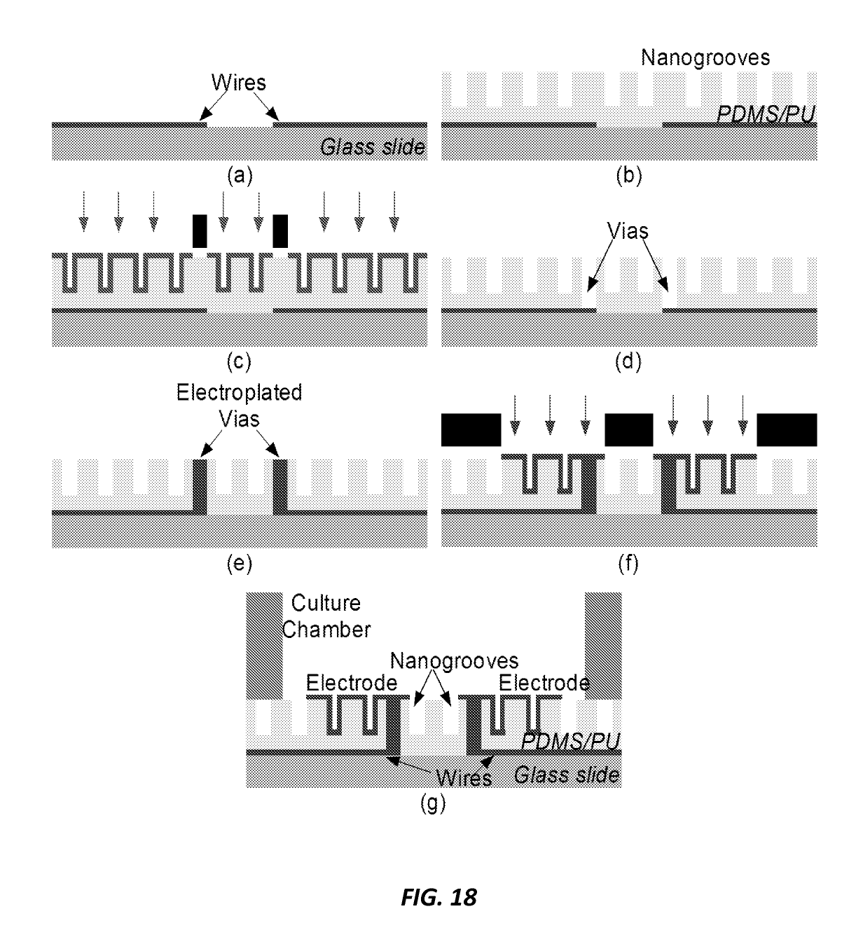

FIG. 18 illustrates an exemplary embodiment of how to form an MEA device having nanogrooves in a batch mode process, which eliminates one by one hand assembly. The process can begin, at FIG. 18(a) with a glass slide and a thin metal film (i.e., Cr/Au) for wires can be deposited/patterned using well known thin film deposition processes. The glass slide helps to reduce parasitic capacitances, thus improving measurement accuracy, and it also allows optical transparency for visualization. At FIG. 18(b), a layer of PDMS or PU can be applied (e.g., by adhesion or casting to the surface of the glass slide. Then, nanogrooves on the PDMS or PU layer, 5-10 μm thick, can be transferred from a mold or stamped on top of the glass slide, using capillary force lithography [2, 3, 20-23]. This transfer or stamp step does not require alignment to the glass slide. Note that once this step is complete, in order to maintain the delicate features of nanogrooves, no high temperature step may be performed. At FIG. 18(c) using a 1^{st} shadow mask, a thin layer of metal can be deposited, i.e., 200 nm of Al, and a reactive ion etch (RIE) process can be used to remove portions of the PDMS/PU using the metal mask. Once etching is complete at FIG. 18(d), the metal mask can be removed and the vias, made of Au, can be formed (e.g., electroplated or deposited) on the bottom metal as shown in FIG. 18(e). After the electroplating step at FIG. 18(f), a 2^{nd} shadow mask can be used to form the electrodes, made of 50-100 nm thick Au, on the contour of the nanogrooves. Finally at FIG. 18(g), the culture chamber can be attached using UV curable epoxy to complete the fabrication.

Evaluation of the Single-Well MEA on Nano-Grooves.

The single-well MEA can be characterized using a sequence of the evaluation protocol. Emulated biopotential signals can be fed at the electrodes of MEA and temporal output signals compared to the emulated ones, as a function of the orientation of nano-grooves. This permits the characterization of the frequency response and minimum detectable signal as a function of nanogroove orientation.

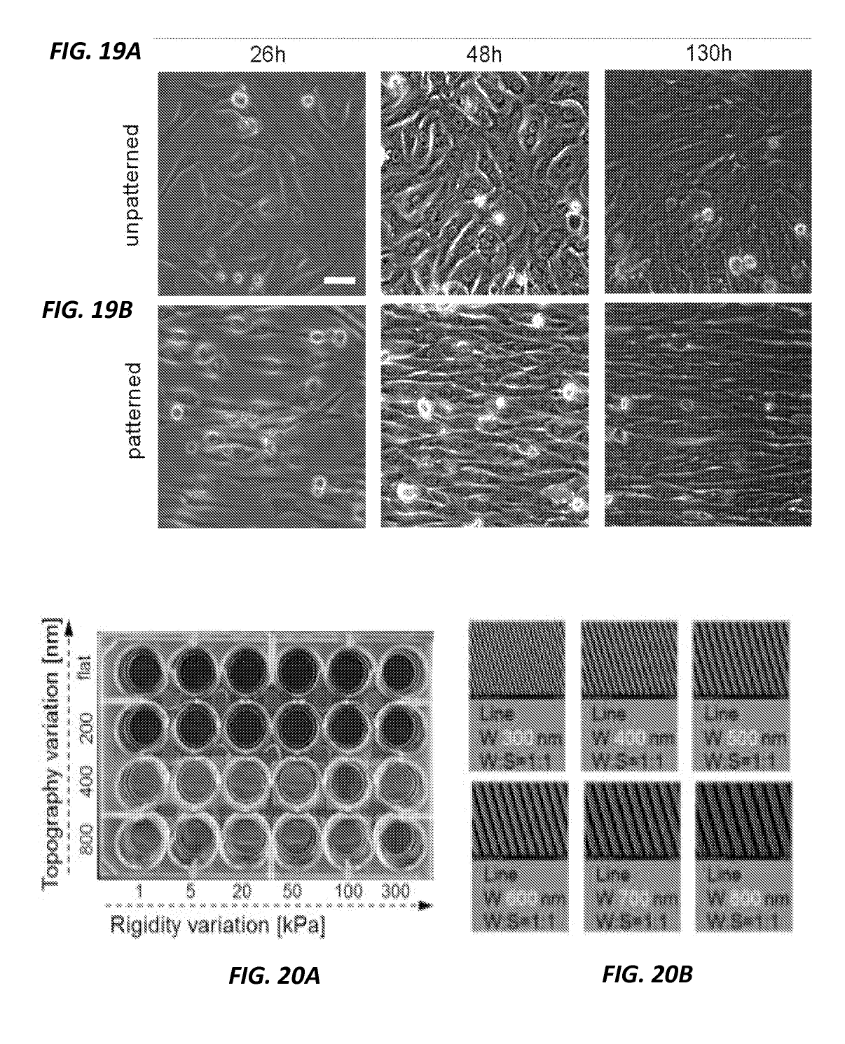

Neonatal ventricular myocytes (NRVMs) can be seeded on the nanopatterned substrates for 6-14 days. As demonstrated herein, cells exhibited near confluence with virtually no intercellular clefts. After 48 hrs, the confluent monolayers formed macroscopically into anisotropic arrays, in contrast to the random orientation of cells in monolayers grown on unpatterned surfaces (FIGS. 19A-19B). To examine the effects of nanogroove parameters (width, pitch, and depth) on structural properties of cardiomyocytes, the angle, length, and width of cardiac cells in monolayer can be measured day by day. For electrophysiological property characterization, NRVMs can be cultured on a nanopatterned MEA device and electrophysiological signals and conduction velocity from cultured monolayers measured at 1 week. It is contemplated herein that aligned nanopatterns can guide the self-assembly of cell monolayers and fast directional propagation of action potential conduction velocity that mimic the mechanoelectrical properties of aligned cardiophysiological architecture of the native heart myocardial tissues.

Discussion.

The acquisition of electrophysiological biosignals as a function of nano-meter scale cues, mimicking in-vivo environments, is useful and has potential to transform the capability of MEAs in biological studies. Unlike conventional MEAs that use a single layer of wires/electrodes, the signal chain of the proposed MEA includes vias that connect the electrodes on the nanogrooves and wires on the glass slide. The contacts between electrodes and vias as well as between vias and wires must be void free to avoid false-positive/negative signals. This can be addressed by in-situ infra-red microscope monitoring during electroplating vias. The infra-red microscope is often used to monitor the quality of vias for electronic circuits. The difference here is that significantly larger-sized vias through polymer rather than high-aspect ratio vias in silicon. By adopting the monitoring protocol the electroplating recipe can be characterized to optimize vias formation.

It is demonstrated herein that rat cardiomyocytes cultured on embodiments of the disclosed nanoogroved device made of PDMS or PU organize into a 2-dimensional mimetic of adult heart tissue. However, nanopatterned PDMS or PU may elicit less adhesion activities of differentiated cardiomyocytes, which prevent cardiac cells to form into a functional, electrically connected monolayer. It is contemplated herein that in some embodiments, the devices described herein can comprise self-assembled adhesion peptides (e.g. RGD) bound to polyurethane-based materials to promote cell-substrate attachment and further augment cellular response.

Multi-Well MEA on Nano-Grooves.

The single-well MEA described above provides useful electrophysiological data of cell growth/proliferation as a function of nanoscale features on a soft material, which mimic in-vivo environments. The orientation and design parameters (width, pitch, and depth) of nanogrooves impact cell growth/proliferation and the acquired multi-channel data may unveil critical aspects of biological study. On the other hand, many applications often demand high throughput, which can be addressed with, e.g., a multi-well MEA on nanogrooves as described herein.

FIGS. 20A-20B depict one embodiment of a multi-well cell culture device having nanogrooved substrates without MEA. It consists of a 24-well plates with each well integrated with a large area nano-topographic substrate. The substrate can be designed with varying rigidity and topography, enabling screening of a different topography/rigidity in each well. Using this high-throughput platform, the structural and functional properties of cardiomyocyte constructs to variable substrate rigidity and topography were rapidly analyzed. Together with the biocompatibility of the material used (i.e. PDMS or PU-based polymer), the scalability of patterns over 5 orders of magnitude (10⁻⁷ m feature size to 10⁻² m substratum size) presents opportunities to directly use the material as a scaffold for construction of implantable engineered myocardial patches. It is contemplated that a similar approach can be utilized to construct a batch-mode multi-well, 96-well, MEA integrated with nanogrooves.

Design and Implementation of One Embodiment of Multi-Well MEA on Nano-Grooves.

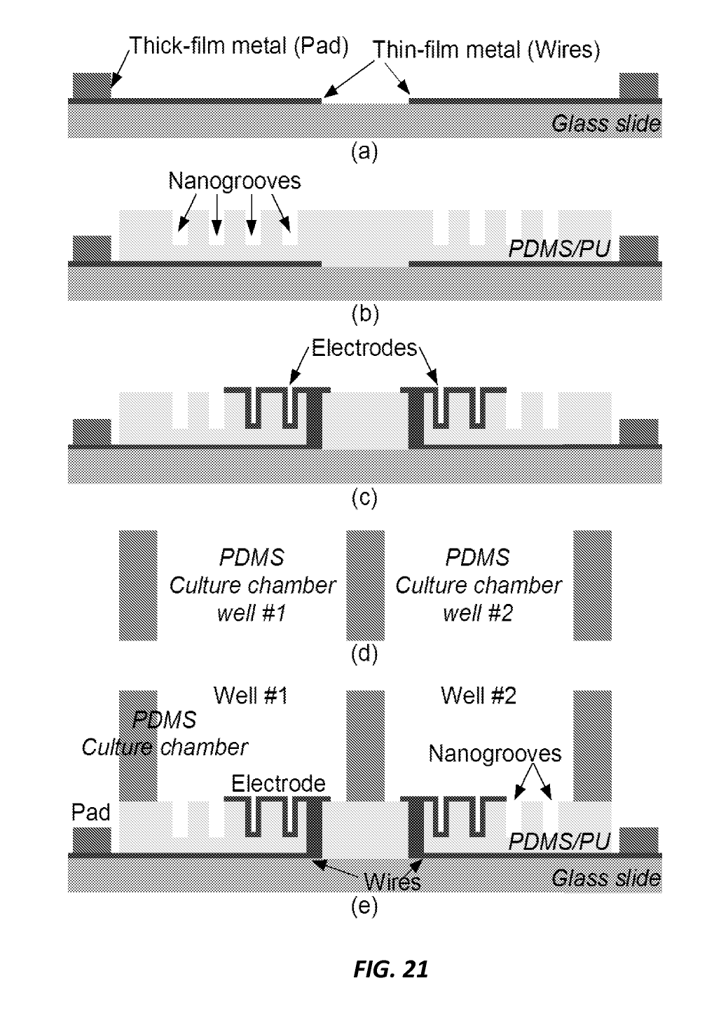

The manufacturing process resembles the one from the single-well MEA, except the interface of the large number of channels (128 channel/well×96 well=12,288 channels) and multiple culture chambers. FIG. 21 depicts the manufacturing sequence of the multi-well MEA. First at FIG. 21(a), wires can be metallized (thin film, 50-100 nm) on a glass slide and then pads can be metallized (thick film, 5-10 μm) for the socket attachment to the leads. After the metallization steps at FIG. 21(b), the PDMS/PU layer can be formed and nanogrooves can be transferred or stamped to the glass slide. Next at FIG. 21(c), electroplate vias can be formed, and electrodes can be formed on top of nanogrooves. These steps are similar to those of a single-well MEA. Once formation of the electrodes is complete, the culture chamber array can be prepared at FIG. 21(d). The chamber array can be molded on PDMS at FIG. 21(e), with a size of up to 12×8 chambers. The dimensions of the chamber array can designed to be in accordance to standard microplates, ˜6.9 mm diameter, ˜10 mm deep and ˜9.0 mm well spacing, which holds a maximum of 320 μL of volume in a chamber. The molded chamber array can be exposed to O₂ plasma, 100 W for 2 mins, and bonded to the glass MEA slide. The total footprint of the 96-well MEA system can be 128×86 mm².

Evaluation of the Multi-Well MEA on Nano-Grooves.

NRVMs can be seeded on the nanopatterned substrates for 6-14 days, similar to the evaluation process of the single-well MEA. To account for the scale of ECM cues and possible variability in the diameter of ECM fibrils in heart tissue, the widths of grooves and ridges in the designed patterns can be varied from 150_{—}50 to 1000_{—}1000 nm and height from 200 to 600 nm. A range of topographical parameters (e.g. width, pitch, and height of grooves/ridges) and an initial cell plating density can be identified to: 1) produce a confluent syncytial monolayer of tri-cell cultures; 2) align cardio-myocytes; 3) allow for detachment of an intact cell monolayer after 1 week of culture. Endpoints can be confluency as evidenced by the ratio of a cytosolic dye area to the total culture area, cardio-myocyte elongation, and variance in cardio-myocyte eccentricity across tissue level. Multi-well platforms integrated with MEA-nanotopographic substrates of varying topographic dimensions in each well can permit rapid analysis of these endpoints. The selected range of nanotopographic dimensions can then be used to generate anisotropic cardiac tissue constructs and characterize nanotopographical effects on cardiomyocyte function and maturation.

Discussion.

The multi-well nanogrooved MEA is an attractive tool to monitor cells in in-vivo like environments. In addition to contacts associated with vias, the multi-well MEA has another metal-metal contact between wires (thin-metal film) and pads (thick-metal film). It is important to maintain signal integrity near the MEA and sintering (300-400° C.) can be performed after forming the contacts. The relatively-low temperature sintering allows re-flow of metal to remove voids and irregular contacts (note that this sintering step is performed before the transfer/stamp of PDMS/PU nanogrooves to be within the strict thermal budget). Cross talk among MEAs from multiple wells is also possible. Unlike a single-well MEA, the signal density of the multi-well MEA is very high. The footprint of a 96-well MEA is 128×86 mm², yet the area for signal routing is fairly limited with a maximum of up to 7.2×10³ mm². When 12,288 signal lines are packed within this limited space, undesirable cross talk can occur. Optimized symmetric layout of the signal lines can be used to reduce the effect of cross talk. The symmetric layout is often used in interfacing peripheral electronics for integrated circuits and a well-developed commercial routing tool will generate the layout. In addition, it is contemplated herein that a ground plane underneath the signal lines can minimize cross talk.

Cardiac maturation is difficult to define through a single endpoint characterization. Cardiac maturation can be marked by ultrastructural organization of the sarcomere and alignment of myofibrils, increased contractile strength, isoform switching between troponin I and titin, increased rate of Ca²⁺ release and uptake, hypertrophy, alignment of the cardio-myocytes, formation of intercalated disks, and increased contractile force generation. Electrophysiological properties of cultured monolayers of cardiac cells on both unpatterned and nanopatterned substrates can be compared. To determine improvements in cell-cell coupling and anisotropic action potential propagation that allows directional contraction of cardiac tissue, biosignals from MEA devices can be utilized and action potential conduction velocity measured for physiological assessment of engineered cardiac cells.

Interface of Biopotentials and Impedance Measurements.

In order to readout biosignals from the MEA, an interface can be used to collect the biosignals in analog, digitize them, and signal-condition them through a standard digital signal processor (DSP). The electronic interface used to collect data from the MEA can be, e.g., a simple single-well single-channel breadboard-level readout, which incorporates an instrumentation amplifier, having a gain of 45, connected to a 4^{th} bandpass gain stage with input high pass (G₂=91, BW=0.05 Hz-1,061 Hz), yielding total gain of 4,100. The amplified/filtered signal can then be connected to DAQ to digitize the signal and visualized in a LabVIEW. The sampling rate can be set to 100 kHz.

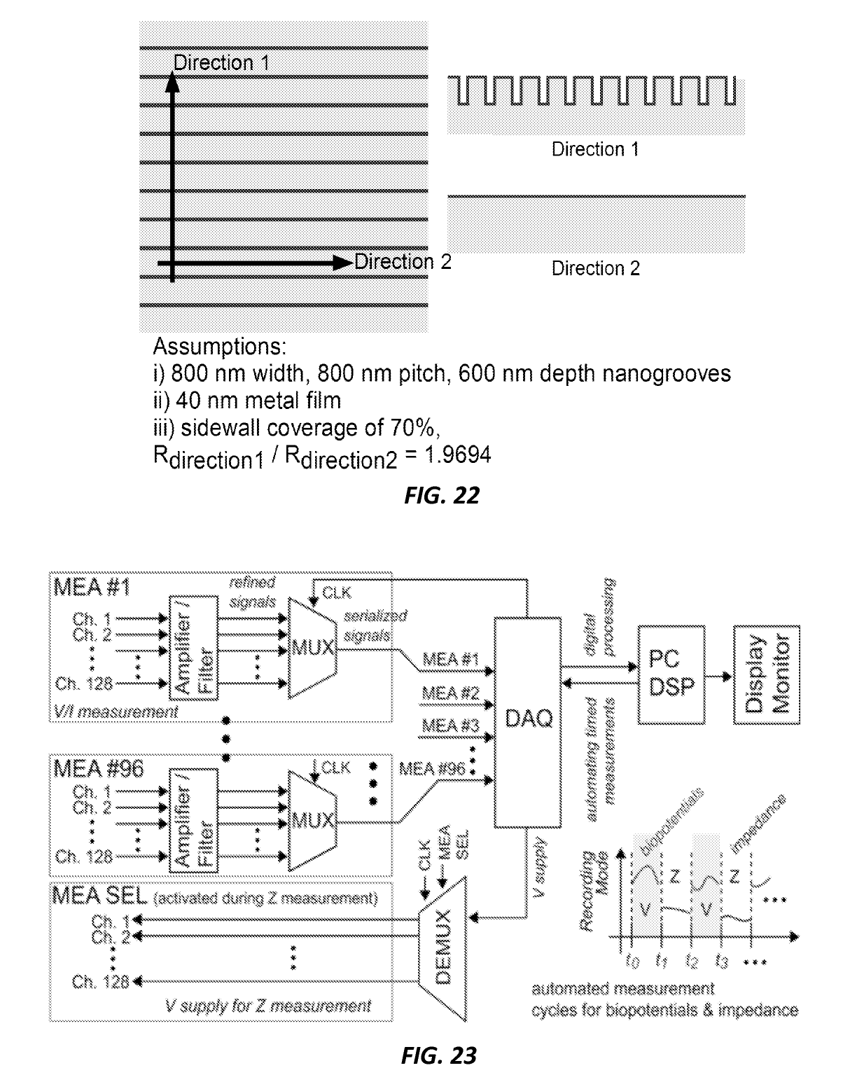

When biosignals are collected at the electrode and transferred via wires, the propagation delay through the wires is dependent on the orientation of nanogrooves. FIG. 22 shows two extreme cases of signal transfer: one is orthogonal (direction 1) and the other is in parallel to the nanogrooves (direction 2). As the effective length of a wire in direction 1 is much longer (1.96×) than that in direction 2, the RC time constant of direction 1 is 3.84 (=1.96²) larger than that of direction 2. For a single-well MEA, the variation of RC time constant has no impact on the measurement as individual channels are collected independently.

However, when biosignals are colelcted from multi-well using time multiplexing method the impact of RC time constant variation becomes significant. In the worst-case scenario, the RC time constant variation is 1.96×. Suppose the RC time constant of the 128 wires for a single well were to be averaged, that factor reduces to 1.614, which yields detection uncertainty of 2.6 times (=1.614²) among the 128 channels, unless a tedious algorithm is implemented to accommodate the delay of individual channels. Alternatively, it is contemplated herein that these wires can be transferred from the top nanogrooved surface to the flat (unpatterned) surface (FIGS. 18 and 21).

Biosignals (Biopotentials and Impedance) Measurements.

The main biosignal of interest is biopotentials from cells. Each electrode on the MEA forms a channel and the biopotential recorded from this electrode is amplified, filtered, and digitized. The other biosignal of interest is impedance. When cells sit on electrodes, the local ionic environment changes, leading to an increase in the electrode impedance. The impedance is a function of the number of cells and quality of cell interaction with the electrodes, including cell adhesion or spreading. Monitoring both biopotentials and impedance permits monitoring of cell viability, number, and morphology in many assays. In some embodiments, the readout of both biopotentials and impedance can be performed.

Serial/Parallel Data Acquisition Interface.

In some embodiments as shown in FIG. 23, each well can include up to 128 channels or electrodes whose signals are processed through time multiplexing to convert the parallel channels to a serial signal on a single wire. The reason for multiplexing all channels of an individual well is to minimize the number of signals or data paths that the computer can retrieve simultaneously. The signals from each electrode can be processed by an amplifier and/or a filter and connected to a multiplexer (MUX) that provides a single output signal (MEA #1-MEA#96) to the Data Acquisition device (DAQ) that can be connected to a computer (PC) or signal processor (DSP). If each channel had its own parallel signal path, the computer would need to read from 128×96=12,288 signals simultaneously. On the other hand, by reducing the number of parallel paths by time division multiplexing, only one data path is needed from each MEA. This is possible as biosignals are far slower (up to 1 Os of kHz) than modern electronics. Signals from all channels can be amplified and/or filtered before going through a multiplexer (MUX) for time multiplexing. The serialized signals from multiple wells can be digitized in parallel, and then further refined through digital signal processor (DSP). Both biopotentials and impedance (Z) can be captured through the same signal paths, except that impedance measurement can involve the application of external stimuli (AC voltage). DAQ can generate the stimulus voltage, which can be demuxed to provide analog AC voltage to individual channels (electrodes) to compute impedance (Z) by measuring AC current to be ratioed. The time division multiplexing allows pseudo-real-time acquisition of biosignals. Each MEA well can be processed in parallel such that the DAQ retrieves signals simultaneously from the 96 wells.

Discussion.

The interface of a single-well MEA is relatively simple as individual channels are collected, amplified, filtered, digitized, and visualized all independently or in other words, in parallel. In a multi-well MEA, noise due to quantization or time delay caused by multiplexing can occur. Unlike simple time multiplexing of biopotentials, impedance measurements can use external stimuli, that is, an AC voltage, to measure AC current to acquire impedance at a given frequency. The external stimuli need to be demuxed to individual channels, which then collect AC current. Preferably this step can be synchronized and accurately fit to the time multiplexing sequence of serialized signals of individual wells. If this step is not fully synchronized, the accuracy of the time multiplexed signals can be degraded. In some embodiments, the time multiplexing can be addressed by an algorithm, e.g., an algorithm embedded in LabVIEW Signal Express. Individual channels can have checkpoints to ensure the synchronization before multiplexing, which monitors the offset/error of non-perfectly synchronized signals.

Cross talk, or coupling, in multiple well embodiments. The parallel wires from each well to the DAQ can experience unwanted noise due to cross talk. This cross talk often limits the credibility of measurements, especially true when collecting large number of signals (note that 12,288 signals can originate from 96 well MEA). Cross talk in analog signals is much more severe than that in digital signals, thus it is critical to address the cross talk at the very front end of MEA interface. A shielding layer can be included beneath the wires, which are located between nanogrooves PDMS/PU substrate and glass slide. The shielding layer can be made of, e.g., a thin film metal, i.e., 100-200 nm aluminum, and an insulation layer, i.e., 50-100 nm SiO₂ or Si₃N₄, needs to be on top of the shielding layer. Such shielding is proven to be very effective and is often used in integrated circuits, called boot strapping, to minimize noise among multiple signal lines. To address cross talk in digital signals the MEA can be packaged in a shielded environment [25].

Described herein is an MEA device that includes a nanogrooved surface that significantly promotes cardiomyocyte (heart cell) maturation and aligns the myocytes into a functional, electrically connected monolayers (tissue). Nano-surface prepared glass slides can be packaged into industry standard MEA “chips”. One embodiment of the single-well MEA chip is 10 mm by 10 mm, and has 128 active electrodes. The MEA chips describe herein can be integrated into this format. The chip provides the life-support for commercially available cardiac cells, for example, from Cellular Dynamics or Axiogenesis and for the introduction of drug solutions over the tissue surface. The chip can then be placed into the MEA reader and the electrical activity of the heart muscle tissue (not individual cells) is measured, recorded, and analyzed.

The devices described herein permit direct screening of pro-arrhythmic effects (the leading cause of cardiotoxicity and drug withdrawal) at the tissue level. The devices described herein can be used in cardiotoxicity screening with, e.g., 1) pro-arrhythmic drugs and 2) a library of small molecule kinase inhibitors with anti-cancer activity that are either known or suspected to have direct cardiotoxic effects (Enzo KI 80 compound library). Endpoints can include (i) action potential duration, (ii) conduction velocity, (iii) spontaneous beating rate, and (iv) reenterant wave patterns. Contemplated herein is a standalone human cardiotoxicity screen platform. Contemplated herein are applications such as evaluation of environmental pollutants or chemical warfare agents.

### REFERENCES

[1] E.-M. Hur, I. H. Yang, D.-H. Kim, J. Byun, Saijilafu, W.-L. Xu, P. R. Nicovich, R. Cheong, A. Levchenko, N. Thakor, and F.-Q. Zhou, “Engineering neuronal growth cones to promote axon regeneration over inhibitory molecules,” Proceedings of the National Academy of Sciences, vol. 108, pp. 5057-5062, Mar. 22, 2011.
[2] D.-H. Kim, K. Han, K. Gupta, K. W. Kwon, K.-Y. Suh, and A. Levchenko, “Mechanosensitivity of fibroblast cell shape and movement to anisotropic substratum topography gradients,” Biomaterials, vol. 30, pp. 5433-5444, 2009.
[3] D.-H. Kim, E. A. Lipke, P. Kim, R. Cheong, S. Thompson, M. Delannoy, K.-Y. Suh, L. Tung, and A. Levchenko, “Nanoscale cues regulate the structure and function of macroscopic cardiac tissue constructs,” Proceedings of the National Academy of Sciences, vol. 107, pp. 565-570, Jan. 12, 2010.
[4] D.-H. Kim, C.-H. Seo, K. Han, K. W. Kwon, A. Levchenko, and K.-Y. Suh, “Guided Cell Migration on Microtextured Substrates with Variable Local Density and Anisotropy,” Advanced Functional Materials, vol. 19, pp. 1579-1586, 2009.
[5] T. Garzon-Muvdi, P. Schiapparelli, C. ap Rhys, H. Guerrero-Cazares, C. Smith, D.-H. Kim, L. Kone, H. Farber, D. Y. Lee, S. S. An, A. Levchenko, and A. Qui√±ones-Hinojosa, “Regulation of Brain Tumor Dispersal by NKCC1 Through a Novel Role in Focal Adhesion Regulation,” PLoS Biol, vol. 10, p. e1001320, 2012.
[6] Kshitiz, M. E. Hubbi, E. H. Ahn, J. Downey, J. Afzal, D.-H. Kim, S. Rey, C. Chang, A. Kundu, G. L. Semenza, R. M. Abraham, and A. Levchenko, “Matrix Rigidity Controls Endothelial Differentiation and Morphogenesis of Cardiac Precursors,” Sci. Signal., vol. 5, pp. ra41-, Jun. 5, 2012.
[7] X. Zhang and J. Chae, “Working Distance Comparison of Inductive and Electromagnetic Couplings for Wireless and Passive Underwater Monitoring System of Rinsing Process in Semiconductor Facilities,” Sensors Journal, IEEE, vol. 11, pp. 2932-2939, 2011.
[8] X. Zhang, J. Yan, B. Vermeire, F. Shadman, and J. Chae, “Passive Wireless Monitoring of Wafer Cleanliness During Rinsing of Semiconductor Wafers,” Sensors Journal, IEEE, vol. 10, pp. 1048-1055, 2010.
[9] H. N. Schwerdt, F. A. Miranda, and C. Junseok, “A Fully Passive Wireless Backscattering Neurorecording Microsystem Embedded in Dispersive Human-Head Phantom Medium,” Electron Device Letters, IEEE, vol. 33, pp. 908-910, 2012.
[10] H. N. Schwerdt, X. Wencheng, S. Shekhar, A. Abbaspour-Tamijani, B. C. Towe, F. A.
Miranda, and C. Junseok, “A Fully Passive Wireless Microsystem for Recording of Neuropotentials Using RF Backscattering Methods,” Microelectromechanical Systems, Journal of, vol. 20, pp. 1119-1130, 2011.
[11] C. A. Thomas Jr, P. A. Springer, G. E. Loeb, Y. Berwald-Netter, and L. M. Okun, “A miniature microelectrode array to monitor the bioelectric activity of cultured cells,” Experimental Cell Research, vol. 74, pp. 61-66, 1972.
[12] I. Jones, P. Livi, M. Lewandowska, M. Fiscella, B. Roscic, and A. Hierlemann, “The potential of microelectrode arrays and microelectronics for biomedical research and diagnostics,” Analytical and Bioanalytical Chemistry, vol. 399, pp. 2313-2329, Mar. 1, 2011.
[13] A. Stett, U. Egert, E. Guenther, F. Hofmann, T. Meyer, W. Nisch, and H. Haemmerle,
“Biological application of microelectrode arrays in drug discovery and basic research,” Analytical and Bioanalytical Chemistry, vol. 377, pp. 486-495, Oct. 1, 2003.
[14] M. Systems. MEA-System [Online]. Available on the world wide web at http://www.multichannelsystems.com/downloads/documentation
[15] Q. Biosciences. MEA60 Biochip Products [Online]. Available Available on the world wide web at http://www.qwane.com/Products/MEAs/MEAs.html
[16] A. M. Scientific. MED64 System [Online]. Available Available on the world wide web at http://www.med64.com/products.html
[17] A. Biosystems. Planar Electrode Arrays [Online]. Available Available on the world wide web at http://www.axionbiosystems.com/wp-content/uploads/2012/05/Singlewell-M64-GLx_datasheet.pdf
[18] A. Bershadsky, M. Kozlov, and B. Geiger, “Adhesion-mediated mechanosensitivity: a time to experiment, and a time to theorize,” Current Opinion in Cell Biology, vol. 18, pp. 472-481, 2006.
[19] F. Rehfeldt, A. J. Engler, A. Eckhardt, F. Ahmed, and D. E. Discher, “Cell responses to the mechanochemical microenvironment, ÄîImplications for regenerative medicine and drug delivery,” Advanced Drug Delivery Reviews, vol. 59, pp. 1329-1339, 2007.
[20] D.-H. Kim, P. Kim, I. Song, J. M. Cha, S. H. Lee, B. Kim, and K. Y. Suh, “Guided Three-Dimensional Growth of Functional Cardiomyocytes on Polyethylene Glycol Nanostructures,” Langmuir, vol. 22, pp. 5419-5426, Jun. 1, 2006.
[21] P. Kim, D. H. Kim, B. Kim, S. K. Choi, S. H. Lee, A. Khademhosseini, R. Langer, and K. Y. Suh, “Fabrication of nanostructures of polyethylene glycol for applications to protein adsorption and cell adhesion,” Nanotechnology, vol. 16, p. 2420, 2005.
[22] K. Y. Suh, H. E. Jeong, K. Deok-Ho, R. A. Singh, and E.-S. Yoon, “Capillarity-assisted fabrication of nanostructures using a less permeable mold for nanotribological applications,” Journal of Applied Physics, vol. 100, pp. 034303-034303-5, 2006.
[23] E. S. Yoon, R. A. Singh, H. Kong, B. Kim, D. H. Kim, H. E. Jeong, and K. Y. Suh, “Tribological properties of bio-mimetic nano-patterned polymeric surfaces on silicon wafer,” Tribology Letters, vol. 21, pp. 31-37, Jan. 1, 2006.
[24] H. M. Himmel, “Drug-induced functional cardiotoxicity screening in stem cell-derived human and mouse cardiomyocytes: Effects of reference compounds,” Journal of Pharmacological and Toxicological Methods, vol. 68, pp. 97-111, 2013.
[25] C.-K. K. Yang, “Design of High-Speed Serial Links in CMOS,” 1998.
[26] S. Je, N. Wang, H. Braun, D. Arnold, and J. Chae, “An Electromagnetically Actuated Microspeaker with Fully-integrated Wax-bonded NdFeB Micromagnets for Hearing Aid Applications,” in International Conference on Solid-state Sensors and Actuators, Denver, Colo., 2009, pp. 885-888.
[27] S.-S. Je, J. C. Harrison, M. N. Kozicki, B. Bakkaloglu, S. Kiaei, and J. Chae, “In situ tuning of a MEMS microphone using electrodeposited nanostructures,” Journal of Micromechanics and Microengineering, vol. 19, p. 035015, 2009.
[28] S.-S. Je, J. Kim, J C Harrison, M. N. Kozicki, and J. Chae, “In-situ tuning of omnidirectional microelectromechanical-systems microphones to improve performance fit in hearing aids,” Applied Physics Letters, vol. 93, p. 123501, 2008.
[29] National Society of Professional Engineers.
[30] A. Abbaspour-Tamijani, M. F. Farooqui, B. C. Towe, and J. Chae, “A miniature fully-passive microwave back-scattering device for short-range telemetry of neural potentials,” in Engineering in Medicine and Biology Society, 2008. EMBS 2008. 30th Annual International Conference of the IEEE, 2008, pp. 129-132.
[31] M. F. Farooqui, J. Chae, B. Towe, and A. Abbaspour-Tamijani, “An experimental verification of a microwave backscatter tag for recording neural signals,” in IEEE Antennas and Propagation Society International Symposium 2008, pp. 1-4.
[32] W. Xu, X. Zhang, H. Yu, A. Abbaspour-Tamijani, and J. Chae, “In-Liquid Quality Factor Improvement for Film Bulk Acoustic Resonators by Integration of Microfluidic Channels,” IEEE Electron Device Letters, vol. 30, pp. 647-649, 2009.
[33] S. Choi and J. Chae, “A microfluidic biosensor based on competitive protein adsorption for thyroglobulin detection,” Biosensors and Bioelectronics, vol. 25, pp. 118-123, 2009.
[34] S. Choi, R. Wang, A. Lajevardi-Khosh, and J. Chae, “Using competitive protein adsorption to measure fibrinogen in undiluted human serum,” Applied Physics Letters, vol. 97, pp. 253701-3, 2010.
[35] S. Choi, Y. Yang, and J. Chae, “Surface plasmon resonance protein sensor using Vroman effect,” Biosensors and Bioelectronics, vol. 24, pp. 893-899, 2008.
[36] Y. Yang, S Kim, and J. Chae, “An Escherichia Coli Concentrator Using Magnetic Particles in a Microfluidic Channel for the Urinary Tract Infection (UTI) Application,” in Solid-state Sensors, Actuators, and Microsystems Workshop, Hilton head island, 2010, pp. 162-165.

### Example 2

### Creation of Polymeric Nanogrooves on Glass Substrates

Polymeric nanogrooves can be made from a variety of materials, including PDMS, PUA, or other biocompatible hydrogels. To form anisotropic nanogrooves any method for nanopatterning can be used. To create one embodiment of a MEA nanodevice as described herein, UV assisted capillary lithography was used, but any other method of nanopatterning can be potentially used. In one embodiments, glass coverslips can be rinsed with isopropyl alcohol in an ultrasonic bath for 30 min and dried in a stream of nitrogen. An adhesion promoter is typically first spin coated to form a thin layer (˜200 nm) onto the glass coverslips prior to dispensing of the precursor solution. A small amount (˜0.1 ml-0.5 ml) of precursor polymer solution can be drop dispensed onto the glass coverslips, and a PUA mold directly placed on the surface. The precursor can be spontaneously drawn into the surface. The precursor can be spontaneously drawn into the cavity of the mold by means of capillary action and was cured by exposure to UV for ˜30 sec. After curing, the mold can be peeled from the substratum by using a sharp tweezers.

Designing Electrode-on-Nano MEA Nanodevices by Integrating MEAs on Nanotextured Substrates.

A shadow mask can be used for MEA, followed by sputtering of Cr/Au of 40-70 nm through the shadow mask on the nanogrooves. Any other electro conductive material can be used to create electrodes. The number of electrodes in an array can range from 2×2 to 3×6, 8×8, 16×16, or any other arbitrary density. Design can be arbitrarily achieved by appropriate design of the shadow mask. The MEA is integrated with a flexible biocompatible layer, i.e., Polydimethylsiloxane (PDMS) or Polyurethane (PU), that possesses nano-meter scale grooves. The nanogrooves can be 100s of nm in width, pitch, and depth, providing mechanical cues to the cells on top of the grooves. Electrical wires can be embedded between the PDMS or PU layer and glass slide. The electrodes can be on the nanogrooves and vias can be formed through the PDMS or PU layer to connect the electrodes and wires. The electrodes can follow the contour of nanogrooves, which allow seamless interface between cells and the nano-scale in-vivo mimicking environment. The glass slide allows fluorescent visualization and helps lower parasitics associated with electrical wires.

Creating Electrode-on-Nano MEA Nanodevice.

In some embodiments, nanogrooves are on PDMS, or other biocompatible hydrogels, which largely limits the thermal budget of subsequent steps. Further, where electrodes/leads are conformal to the nanogroove profile, the resistance is a function of orientation of nanogrooves. The measured resistances of leads aligned parallel and perpendicular to the nanogrooves are 10-13Ω and 20-27Ω, respectively, matching well with the geometrical calculation. In order to provide electrical isolation among MEA electrodes, a thin (˜300 nm) biocompatible insulator, parylene-C, can be deposited on top of the electrical leads, leaving the electrodes uncovered. The deposition process can be performed at room temperature to preserve the delicate features of the nanogrooves. The parylene-C layer can cover leads and electrodes can be uncovered to allow for exposure to cells. After the deposition, a culture chamber can be attached on the chip using a UV-curable biocompatible adhesive (PU 78).

Design and Implementation of Single Well MEA Nanodevice.

MEA Nanodevices as described herein can be created in batch mode process, as well as by hand assembly for low throughput. In one embodiment, starting from a glass slide, a thin metal film (i.e., Cr/Au) for wires is deposited/patterned. The glass slide helps to reduce parasitic capacitances, thus improving measurement accuracy, and it also allows optical transparency for visualization. Then, nanogrooves on the PDMS or PU layer, 5-10 μm thick, can be transferred from a mold or stamped on top of the glass slide, using, e.g., capillary force lithography. This transfer or stamp step does not require alignment to the glass slide. Note that once this step is complete, in order to maintain the delicate features of nanogrooves, no high temperature step may be performed. Using 1^{st} shadow mask a thin layer of metal, i.e., 200 nm of Al, is deposited and reactive ion etch (RIE) PDMS/PU is performed using the metal mask. Once etching is complete, the metal mask is removed and the vias, made of Au, can be electroplated from the bottom metal. After the electroplating step, a 2^{nd} shadow mask can be used to put down electrodes, made of 50-100 nm thick Au, on the contour of the nanogrooves. Finally, the culture chamber can be attached using UV curable epoxy to complete the fabrication. This method can be modified to use other nanopatterning and sputtering techniques.

Evaluation of MEA Nanodevice.

A single-well MEA can be characterized using a sequence of the evaluation protocol. Feed emulated biopotential signals at the electrodes of MEA and compare temporal output signals to the emulated ones, as a function of the orientation of nano-grooves By doing so, the frequency response and minimum detectable signal can be characterized as a function of nanogroove orientation.

For further evaluation, neonatal ventricular myocytes (NRVMs) can be seeded on the nanopatterned substrates for 3 days. As described herein, cells exhibited near confluence with virtually no intercellular clefts. After 48 hrs, the confluent monolayers formed macroscopically into anisotropic arrays, in contrast to the random orientation of cells in monolayers grown on unpatterned surfaces. To examine the effects of nanogroove parameters (width, pitch, and depth) on structural properties of cardiomyocytes, the angle, length, and width of cardiac cells in monolayer can be measured day by day. For electrophysiological property characterization, NRVMs can be cultured on nanopatterned MEA device and electrophysiological signals and conduction velocity from cultured monolayers measured at 1 week. Other cardiac cell types can be used. The design of MEA Nanodevice can be arbitrarily designed to fit with any commercial (or home made) amplifiers, allowing for direct measurements of biopotential signals using established MEA protocols.

Creating a Multi-Throughput MEA Nanodevice.

To create a multi-throughput electrode-on-nano MEA Nanodevice, one embodiment contemplated herein is hybrid integration of an array of wells with the nano-manufactured array of MEAs. When the array becomes large, for example, up to 128 channels per well and 96 wells, the total number of channels becomes enormous, that is, 128×96=12,288. It is very challenging to collect signals from all of these channels and retrieve “real-time” like signals. This data can be collected, in some embodiments, by constructing a systematic interface, permitting pseudo real-time acquisition of biosignals. The manufacturing process resembles the one from the single-well MEA Nanodevice, except the interface of the large number of channels (128 channel/well×96 well=12,288 channels) and multiple culture chambers. FIG. 21 shows the manufacturing sequence of this embodiment of multi-well MEA. First, wires can be metalized (thin film, 50-100 nm) on a glass slide and then pads are metalized (thick film, 5-10 μm) for the socket attachment at the end. After the metallization steps, transfer or stamp nanogrooves to the glass slide, electroplate vias, and form electrodes on top of nanogrooves. These steps are very similar to those of a single-well MEA.

Integrating Large Surface Area MEA Nanodevice with a Multiwell Chamber.

Once complete formation of the electrodes is achieved, the culture chamber array can be prepared. In some embodiments, the chamber array can be molded on PDMS, with a size of up to 12×8 chambers. The dimensions of the chamber array can be designed to be in accordance to standard microplates, ˜6.9 mm diameter, ˜10 mm deep and ˜9.0 mm well spacing, which holds a maximum of 320 μL of volume in a chamber. The molded chamber array can be exposed to O₂ plasma, 100 W for 2 mins, and bonded to the glass MEA slide. The total footprint of the 96-well MEA system can be 128×86 mm².

The chamber can be directly fabricated by injection molding, or commercially procured and adhered to the large surface area MEA Nanodevice by any of the variety of biocompatible adhesion methods (UV curable adhesive, thermally curable adhesive etc). FIG. 21 explains the protocol.

### Example 3

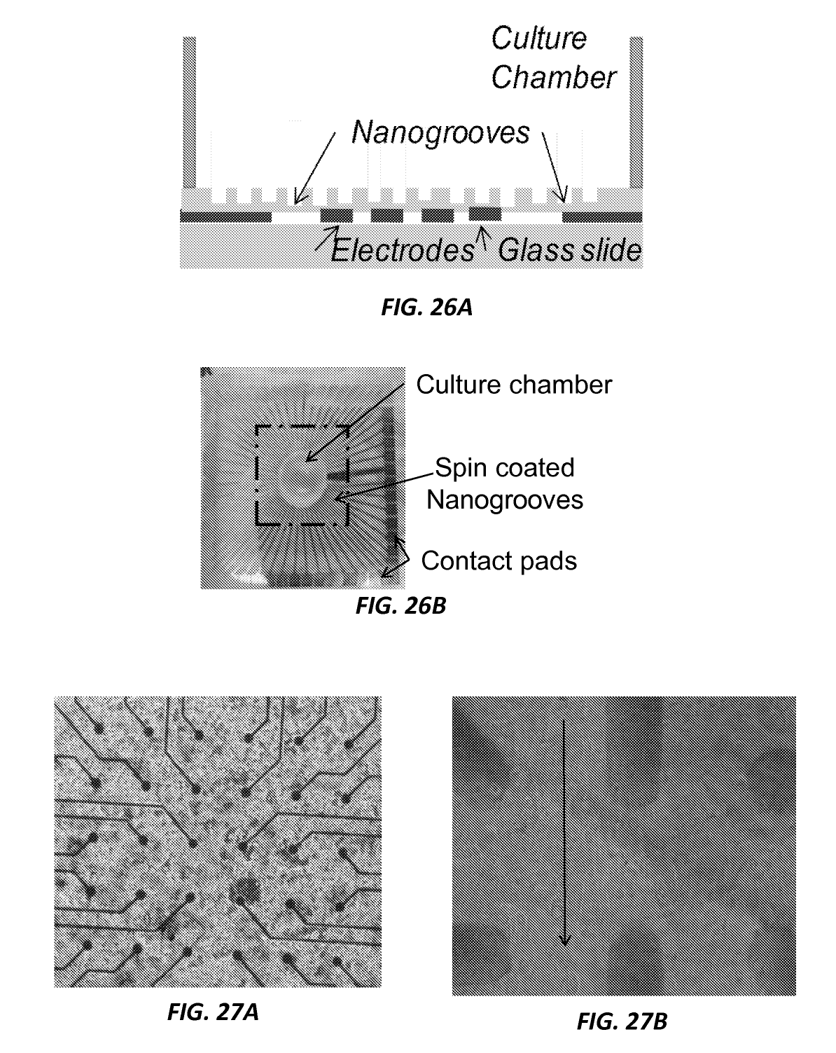

Cardiomyocytes readily form monolayers on MEA nanodevices (FIG. 27A-27B). Devices with electrodes above or below the nanogrooves were coated with extracellular matrix molecules and/or synthetic peptides. Cardiomyocytes seeded on the devices then formed monolayers aligned with the direction of the nanogrooves.

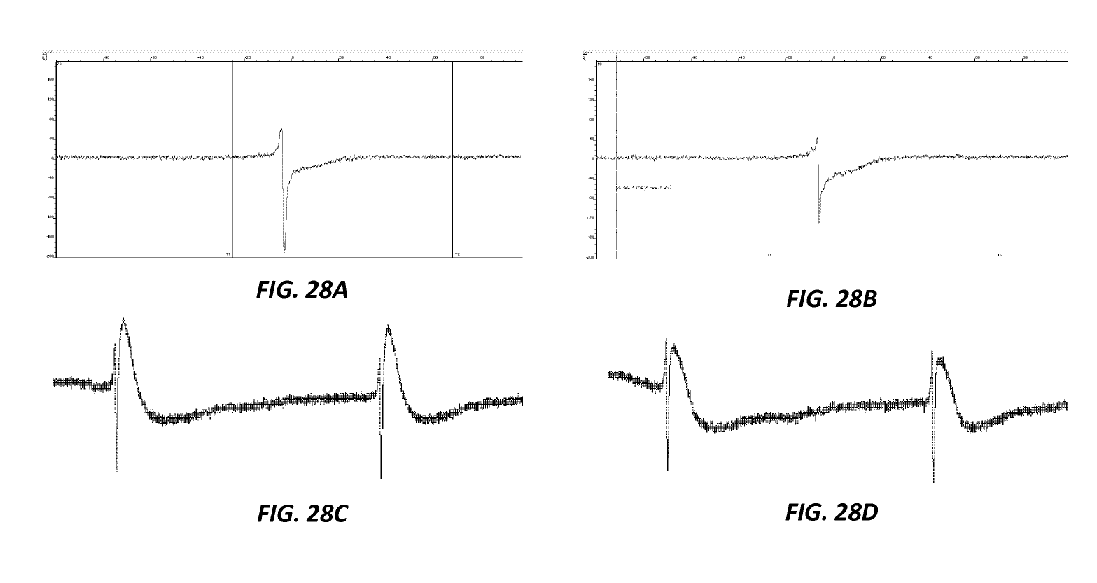

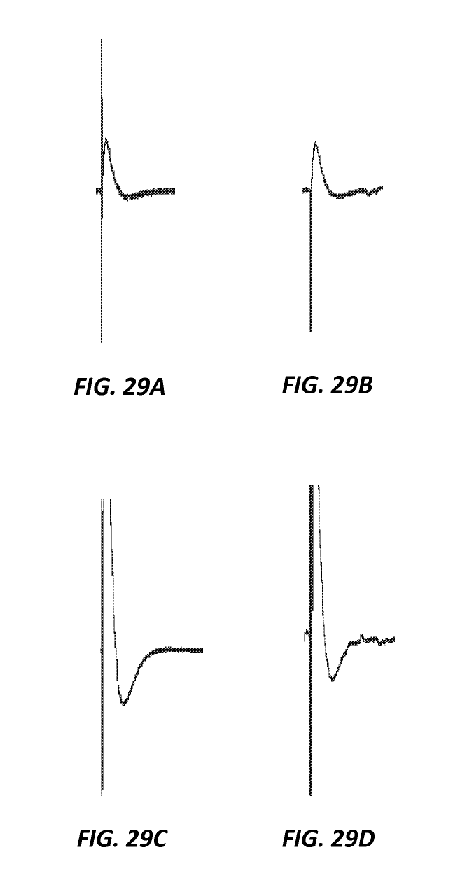

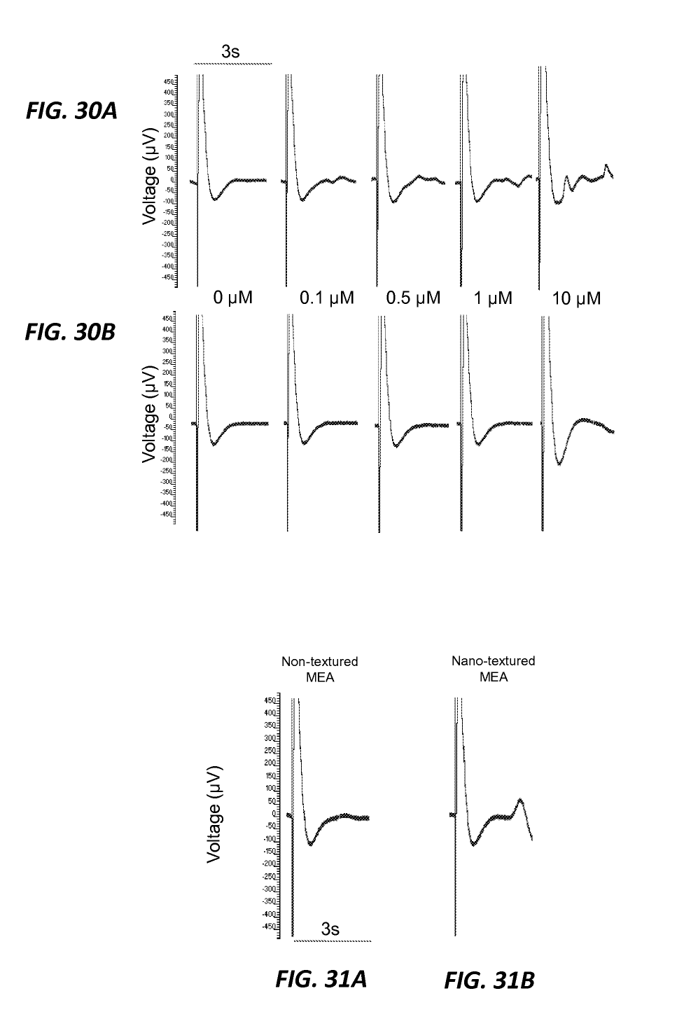

Spontaneously generated field potentials of rat myocytes and human cardiomyocytes were not altered by the nantoextured MEA devices (FIG. 28A-28D). Additionally, the devices described herein were more sensitive in detect effect of drugs on cardiac activity on cells (FIGS. 29A-29B, FIGS. 30A-30B).

A MEA nanodevice as described herein can accurately identify drugs that are known false positives (FIGS. 30A-30B). Verapamil, a known hERG inhibitor, causes dose dependent aberration in field potential profiles of human pluripotent stem cell derived cardiomyocytes cultured on non-textured MEAs (FIG. 30A), but does not cause detectable aberration in MEA nanodevice (FIG. 30B).

MEA nanodevices as described herein are more sensitive in detecting effect of drugs on cardiac activity on cells matured and anisotropically aligned over the nanotextured platform. (FIG. 31A) Addition of 1 μM Calcium channel inhibitor causes detectable aberration in field potential profiles of human pluripotent stem cell derived cardiomyocytes cultured over non-textured (unpatterned) MEAs. Abberrations in field potential profiles were detected at significantly lower doses of the inhibitor in cells grown on nanotextured devices (at 0.02 μM cisapride) versus cells grown on non-textured MEAs (FIG. 31B).

1. A substrate comprising a plurality of microelectrodes and electroconductive cells, wherein the microelectrodes contact the electroconductive cells, and wherein the microelectrodes are connected to an electronic interface to transmit a signal input to the cells or record the electrical signals from the cells.

2.-15. (canceled)

16. The substrate of claim 1, wherein the electroconductive cells are muscle cells.

17. The substrate of claim 1, wherein the muscle cells are selected from cardiomyocytes, skeletal muscle myocytes, and smooth muscle myocytes.

18. The substrate of claim 1, wherein the electroconductive cells are neuronal cells.

19. The substrate of claim 1, wherein the substrate has a substantially smooth surface.

20. The substrate of claim 1, wherein the substrate has a nanotextured surface.

21. The substrate of claim 20, wherein the nanotextured surface comprises a substantially parallel array of grooves and ridges.

22. The substrate of claim 21, wherein the depth of the grooves is between 10 nm-10 μm, the width of the grooves is between 50 nm-10 μm, and the ridges between the grooves have a width of between 50 nm-10 μm.

23. The substrate of claim 22, wherein the depth of the grooves is between 50 nm-500 nm, the width of the grooves is between 200 nm-1000 nm, and the ridges between the grooves have a width of between 200 nm-1000 nm.

24. A method for disease modeling comprising using the substrate of claim 1 in which the cells are derived from a subject with a muscular disease or disorder.

25. An assay for assessing the effect of an agent on at least one biopotential or electrical property of a population of electroconductive cells, the assay comprising culturing the population of electroconductive cells on the substrate of claim 1, contacting the electroconductive cells with at least one agent, and measuring at least one of: the growth; viability; and a biopotential or electrical property of the population of electroconductive cells.

26. The assay of claim 25, wherein the effect is a toxic or aberrant effect on at least one of the biopotentials or electrical properties of the electroconductive cells.

27. The assay of claim 25, wherein the at least one biopotential of the electroconductive cells is measured on the surface of the microelectrode array.

28. A system comprising:
(a) a substrate comprising microelectrodes, wherein the microelectrodes on the substrate are connected to an electronic interface,
(b) an electronic interface which is connected to a computer
(c) a computer, wherein the computer instructs the electronic interface for the transmission of electric signals to and from the microelectrodes on the substrate.

29. The system of claim 14, wherein the substrate comprising microelectrodes comprises electroconductive cells.

30. The substrate of claim 1, wherein the electroconductive cells are arranged in an anisotropic and polarized cell arrangement.

31. The substrate of claim 1, further comprising a shielding layer below the microelectrodes, where the shielding layer comprises a thin film metal and an insulating layer.

32. The assay of claim 11, wherein the electroconductive cells are selected from the group consisting of: neuronal cells; cardiomyocytes; skeletal muscle myocytes; smooth muscle myocytes; or a combination thereof.

33. The assay of claim 11, wherein the electroconductive cells are obtained from, or descended from cells of a healthy human subject, or a human subject with a cardiac disease.

34. The assay of claim 11, wherein at least one electrical property of the electroconductive cell is electrical impedance.
